(19) United States
(12) Patent Application Publication
(10) Pub. No.: US 2024/0377411 A1
(43) Pub. Date: Nov. 14, 2024

(54) METHODS FOR DIAGNOSING AN AUTISTIC SPECTRUM DISORDER

(71) Applicant: QATAR UNIVERSITY, Doha (QA)

(72) Inventors: Naila RABBANI, Warkwickshire (GB); Paul J. THORNALLEY, Warwickshire (GB); Kashif Mahmood RAJPOOT, Birmingham (GB); Marina MARINI, Bologna (IT); Provvidenza Maria ABRUZZO, Bologna (IT); Alessandra BOLOTTA, Bologna (IT)

(21) Appl. No.: 18/668,007

(22) Filed: May 17, 2024

Related U.S. Application Data
(63) Continuation-in-part of application No. 18/607,313, filed on Mar. 15, 2024.
Application No. 18/607,313 is a division of application No. 16/967,361, filed on Aug. 4, 2020, now Pat. No. 12,044,684.

(30) Foreign Application Priority Data
Feb. 9, 2018 (GB) 1802116.2

(51) Int. Cl. G01N 33/68

(52) U.S. Cl. CPC G01N 33/6848; G01N 2800/30; G01N 2800/52

(57) ABSTRACT

The present application describes a diagnostic method based on detecting trace levels of chemically defined oxidized, nitrated, and glycated amino acid residues in plasma protein, and related oxidized, nitrated, and glycated amino acids in plasma and urine. The present application describes a machine learning approach to distinguish between children with and without ASD.

### CROSS-REFERENCE TO RELATED APPLICATIONS

This application is a Continuation-in-Part of U.S. patent application Ser. No. 18/607,313, filed on Mar. 15, 2024, which is a Divisional application of U.S. patent application Ser. No. 16/967,361, filed on Aug. 4, 2020, which is the 371 U.S. National Stage entry of PCT International Patent Application No. PCT/GB2019/050362, filed on Feb. 11, 2019, which claims the benefit of priority to United Kingdom Patent Application No. 1802116.2, filed on Feb. 9, 2018. The disclosures of the prior applications are hereby incorporated by reference in their entirety.

### TECHNICAL FIELD

The present application relates to methods for diagnosing an autistic spectrum disorder (ASD) comprising the use of an amino acid adduct.

### BACKGROUND

Autism Spectrum Disorders (ASD) are defined as developmental disorders mainly affecting social interactions and range of interests and causing a wide spectrum of other disabilities, such as speech disturbances, repetitive and/or compulsive behaviors, hyperactivity, anxiety and difficulty to adapt to new environments, with or without cognitive impairment. The high heterogeneity of the clinical presentation makes diagnosis of ASD difficult and uncertain, particularly at the early stages of development. Autism affects about 1% of children and typically has higher prevalence in boys (1.5%) than girls (0.5%).

ASD is currently diagnosed based on a clinician's perception of symptoms in the patient. Scores derived from assessment of patient performance in standardized activities are used, including Autism Diagnostic Observation Schedule (ADOS) score, the childhood autism rating scale (CARS), checklist for autism in toddlers (CHAT) and social communication questionnaire (SCQ). However, these assessments are subjective and provide no information on assessment of risk of progression to more severe symptoms nor assessment of response to therapy. The performance of these methods is poor, they have a sensitivity of only 20-70% and 50-80%. They also require expert an experienced healthcare personnel for implementation. There therefore exists a need for improved methods of diagnosing autism in patients.

Currently, there is no clinical chemistry or imaging test for autism. Diagnosis is based on clinical assessment of impaired social interactions and range of interests. Transcriptomic, proteomic and metabolomic profiling have been proposed for diagnosis of ASD, with diagnostic performance judged by an area under-the-curve of receiver operating characteristic (AUROC) plot of 0.73-0.91. However, such legacy methods are of low specificity. Despite these recent developments, there remains a need for an inexpensive and minimally invasive test for diagnosing a subject with ASD with a high degree of sensitivity and specificity.

### SUMMARY

The present application describes a diagnostic method based on detecting trace levels of chemically defined oxidized, nitrated, and glycated amino acid residues in plasma protein, and related oxidized, nitrated, and glycated amino acids in plasma and urine. The present application describes a machine learning approach to distinguish between children with and without ASD.

In some embodiments, a method for diagnosing an autistic spectrum disorder (ASD) is described, said method comprising obtaining a sample from a subject. In some embodiments, the method comprises detecting a concentration of one or more amino acid adducts in the sample obtained from a subject. In some embodiments, the method comprises calculating a probability of the subject having ADS. In some embodiments, the method comprises diagnosing the patient.

In some embodiments, a method for diagnosing an autistic spectrum disorder (ASD) is described comprising detecting the concentration of an amino acid adduct in a sample obtained from a subject, wherein said amino acid adduct is a glycated amino acid adduct, an oxidised amino acid adduct, or a nitrated amino acid adduct. In some embodiments, the method comprises comparing the concentration of the amino acid adduct in the sample with the concentration of the same amino acid adduct in a reference standard. In some embodiments, the method comprises identifying the presence or absence of a concentration difference of said amino acid adduct in the sample relative to the reference standard, wherein the presence or absence of a concentration difference correlates with the presence or absence of ASD.

In some embodiments, a method for determining prognosis of an autistic spectrum disorder (ASD) is described, said method comprising detecting the concentration of an amino acid adduct in a sample obtained from a subject, wherein said amino acid adduct is a glycated amino acid adduct, an oxidised amino acid adduct, or a nitrated amino acid adduct. In some embodiments, the method comprises comparing the concentration of the amino acid adduct in the sample with the concentration of the same amino acid adduct in a reference standard. In some embodiments, the method comprises identifying the presence or absence of a concentration difference of said amino acid adduct in the sample relative to the reference standard, wherein the presence or absence of a concentration difference may correlate with a prognosis of autism.

In some embodiments, a method for diagnosing an autistic spectrum disorder (ASD) is described, said method comprising detecting the concentration of an amino acid adduct in a sample obtained from a subject, wherein said amino acid adduct is a glycated amino acid adduct, an oxidised amino acid adduct, or a nitrated amino acid adduct. In some embodiments, the method comprises classifying the health of the subject based on the concentration of the amino acid adduct detected in the sample with a diagnostic algorithm, wherein the diagnostic algorithm is trained on the corresponding concentration for the same amino acid adduct obtained from a population of subjects having a known disease status, and thereby diagnosing the presence or absence of ASD in the subject.

In some embodiments, a method for treating an autistic spectrum disorder (ASD) is described, said method comprising requesting the performance or obtaining the results of any of the previous embodiments. In some embodiments, the method comprises administering to a subject diagnosed with ASD, a therapy for ASD.

In some embodiments, a method for identifying a therapy suitable for treating ASD is described, said method comprising providing an isolated sample from a subject administered a candidate therapy. In some embodiments, the method comprises detecting the concentration of an amino acid adduct in said sample, wherein said amino acid adduct is a glycated amino acid adduct, an oxidised amino acid adduct, or a nitrated amino acid adduct. In some embodiments, the method comprises determining the relative concentration change of the amino acid adduct by comparing the concentration of the amino acid adduct detected in a previous embodiment with the concentration of the amino acid adduct in an isolated sample from the subject prior to administration of the candidate therapy, wherein the candidate therapy is suitable for treating ASD when a concentration change is detected after administering the candidate therapy, and wherein the candidate therapy is not suitable for treating ASD when a concentration change is not detected after administering the candidate therapy.

In some embodiments, a method for monitoring the efficacy of an ASD therapy is described, said method comprising providing an isolated sample from a patient administered said therapy. In some embodiments, the methods comprise detecting the concentration of an amino acid adduct in said sample, wherein said amino acid adduct is a glycated amino acid adduct, an oxidised amino acid adduct, or a nitrated amino acid adduct. In some embodiments, the method comprises determining the relative concentration change of the amino acid adduct by comparing the concentration of the amino acid adduct detected in any of the previous embodiments with the concentration of the amino acid adduct in an isolated sample from the subject at an earlier timepoint. In some embodiments, the method comprises confirming the presence of efficacy when a concentration change is detected. In some embodiments, the method comprises confirming the absence of efficacy when a concentration change is not detected.

In some embodiments, the method of any of the previous embodiments comprises using a diagnostic algorithm, preferably wherein the diagnostic algorithm is configured to diagnose the presence or absence of ASD based on the concentration of the amino acid adduct detected in the sample, wherein the diagnostic algorithm is trained on the corresponding concentration for the same amino acid adduct in one or more (preferably a plurality of) reference standards. In certain embodiments, the diagnostic algorithm is configured to classify the health of the subject based on the concentration of the amino acid adduct detected in the sample, wherein the diagnostic algorithm is trained on the corresponding concentration for the same amino acid adduct obtained from a population of subjects having known disease status.

In some embodiments, the method comprises use of a glycated amino acid adduct, an oxidised amino acid adduct, a nitrated amino acid adduct, or a combination thereof for diagnosing ASD. In some embodiments, the method comprises determining prognosis of ASD. In some embodiments, the method comprises identifying a therapy suitable for treating ASD. In some embodiments, the method comprises monitoring efficacy of an ASD therapy. In some embodiments, the method comprises using any of the previous embodiments as a feature in an ASD diagnostic algorithm.

In some embodiments, the method or use according to any one of the preceding embodiments comprises a sample selected from one or more of blood, blood plasma, blood plasma ultrafiltrate, urine, blood serum, synovial fluid and/or sputum.

In some embodiments, the method or use according to any one of the preceding embodiments, comprises one or more an amino acid adducts selected from N_{ε}-carboxymethyl-lysine (CML), 3-deoxyglucosone-derived hydroimidazolone (3DG-H), N_{ω}-carboxymethylarginine (CMA), glutamic semialdehyde (GSA), glyoxal-derived hydroimidazolone (G-H1), pyrraline, methylglyoxal-derived hydroimidazolone (MG-H1), N_{ε}-fructosyl-lysine (FL), N_{ε}-(1-carboxyethyl) lysine (CEL), α-aminoadipic semialdehyde (AASA), and methylglyoxal-derived lysine dimer (MOLD).

In some embodiments, the method or use according to any one of the preceding claims comprises an at least two amino acid adducts selected from N_{ε}-carboxymethyl-lysine (CML), 3-deoxyglucosone-derived hydroimidazolone (3DG-H), N_{ω}-carboxymethylarginine (CMA), dityrosine (DT), glutamic semialdehyde (GSA), glyoxal-derived hydroimidazolone (G-H1), pyrraline, methylglyoxal-derived hydroimidazolone (MG-H1), N_{ε}-fructosyl-lysine (FL), N_{ε}-(1-carboxyethyl) lysine (CEL), α-aminoadipic semialdehyde (AASA), and methylglyoxal-derived lysine dimer (MOLD).

In some embodiments, the method or use according to any one of the preceding embodiments comprises detecting whether the concentration of one or more amino acid adducts selected from: CML, CMA, and GSA is increased compared to a reference standard. In some embodiments, the method comprises detecting whether the concentration of one or more amino acid adducts selected from: 3DG-H and G-H1 (free adduct) is decreased, when compared to a non-ASD reference standard. In some embodiments, a decrease in the concentration of the amino acid adducts indicates the presence of ASD.

In some embodiments, the method or use according to any one of the preceding embodiments comprises detecting whether the concentration of one or more amino acid adducts selected from: CML, CMA, and GSA in a sample is increased or remains the same when compared to a reference. In some embodiments, the method comprises detecting whether the concentration of one or more amino acid adducts selected from: 3DG-H and G-H1 (free adduct) is the same or decreased, when compared to an ASD reference standard, and is indicative of the presence of ASD.

In some embodiments, the method or use according to any one of the preceding embodiments comprises detecting whether concentration of two or more amino acid adducts selected from: CML, CMA, DT, and GSA are increased, when compared to a non-ASD reference standard, and is indicative of the presence of ASD.

In some embodiments, the method or use according to any one of the preceding embodiments, comprises detecting whether the concentration of two or more amino acid adducts selected from: CML, CMA, DT, and GSA are the same or increased, when compared to an ASD reference standard, and is indicative of the presence of ASD.

In some embodiments, the method or use according to any one of the preceding embodiments comprises detecting whether the concentration of one or more amino acids adducts selected from: CML, and CMA is increased; and/or the concentration of 3DG-H is decreased, when compared to a non-ASD reference standard, and is indicative of the presence of ASD.

In some embodiments, the method or use according to any one of the preceding embodiments comprises detecting whether the concentration of one or more amino acid adducts selected from: CML and CMA is the same or increased; and/or the concentration of 3DG-H is the same or decreased, when compared to an ASD reference standard, and is indicative of the presence of ASD.

In some embodiments, the method or use according to any one of the preceding embodiments comprises detecting whether the concentration of two or more amino acid adducts selected from: CML, CMA, and DT is increased, when compared to a non-ASD reference standard, and is indicative of the presence of ASD.

In some embodiments, the method or use according to any one of the preceding embodiments comprises detecting whether the concentration of two or more amino acid adducts selected from: CML, CMA, and DT is the same or increased, when compared to an ASD reference standard, and is indicative of the presence of ASD.

In some embodiments, the method or use according to any one of the preceding embodiments comprises detecting whether the concentration of CML and/or CMA is increased when compared to a non-ASD reference standard, and is indicative of the presence of ASD.

In some embodiments, the method or use according to any one of the preceding embodiments comprises detecting whether the concentration of CML and/or CMA is the same or increased when compared to an ASD reference standard, and is indicative of the presence of ASD.

In some embodiments, method or use according to any of the previous embodiments comprises a sample, wherein the sample is a blood sample.

In some embodiments, the method or use according to any one of the preceding embodiments comprises detecting whether the concentration of GSA and/or pyrraline is increased, when compared to a non-ASD reference standard, and is indicative of the presence of ASD.

In some embodiments, the method or use according to any one of the preceding embodiments comprises detecting whether the concentration of GSA and/or pyrraline is the same or increased, when compared to an ASD reference standard, and is indicative of the presence of ASD.

In some embodiments, the method or use of an of the previous embodiments, comprises a sample, wherein the sample is a urine sample.

In some embodiments, the method or use according to any one of the preceding embodiments, comprises detecting an amino acid adduct, wherein the amino acid adduct is detected by stable isotopic dilution analysis liquid chromatography-tandem mass spectrometry reaction monitoring (SRM) mass spectrometry, Western Blot, Enzyme-Linked Immunosorbent Assay (ELISA), liquid chromatography mass spectrometry (LC-MS), reverse phase mass spectrometry, surface enhanced laser desorption ionisation time-of-flight mass spectrometry (SELDI-TOF), matrix assisted laser desorption ionisation time-of-flight mass spectrometry (MALDI-TOF), liquid chromatography-tandem mass spectrometry, isotope dilution mass spectrometry, size permeation (gel filtration), ion exchange, affinity, high performance liquid chromatography, ultra performance liquid chromatography, one-dimensional gel electrophoresis (1-DE), and/or two-dimensional gel electrophoresis (2-DE).

In some embodiments, the method or use according to any one of the preceding embodiments, comprises detecting an amino acid adduct by mass spectroscopy.

In some embodiments, the method or use according to any one of the preceding embodiments, comprises detecting an amino acid adduct by liquid chromatography-tandem mass spectrometry.

In some embodiments, the method or use according to any one of the preceding embodiments, comprises identifying an autistic spectrum disorder, wherein the disorder is selected from one or more of autism, Asperger syndrome, pervasive developmental disorder not otherwise specified (PDD-NOS), and childhood disintegrative disorder.

In some embodiments the method or use according to any of the preceding embodiments, further comprises a step of recording on a suitable data carrier, the data obtained in the step of detecting the concentration of an amino acid adduct in a sample.

In some embodiments, the method according to any one of the preceding embodiments, comprises entering the concentration of the detected an amino acid adduct into a diagnostic algorithm. In certain embodiments, the diagnostic algorithm indicates whether ASD is present or absent in a subject.

In some embodiments, the method comprises a data carrier comprising the data obtained in the step of detecting the concentration of an amino acid adduct in a sample according to the method or use according to any one of the preceding claims. In some embodiments, the method comprises a data carrier for use in a method for diagnosing an autistic spectrum disorder.

In some embodiments, a kit is described comprising reagents for detecting the concentration of an amino acid adduct in a sample, wherein said amino acid adduct is one or more selected from a glycated amino acid adduct, an oxidized amino acid adduct, a nitrated amino acid adduct or a combination thereof; and instructions for use of the same.

In certain embodiments, the kit comprises an amino acid adduct selected from: N_{ε}-fructosyl-lysine (FL), glyoxal-derived hydroimidazolone (G-H1), 3-deoxyglucosone-derived hydroimidazolone (3DG-H), N_{ε}-carboxymethyl-lysine (CML), N_{ω}-carboxymethylarginine (CMA), methylglyoxal-derived hydroimidazolone (MG-H1), pyrraline, methylglyoxal-derived lysine dimer (MOLD), N-formylkynurenine (NFK), α-aminoadipic semialdehyde (AASA), glutamic semialdehyde (GSA) and 3-Nitrotyrosine (3-NT).

In some embodiments, the kit comprises two or more amino acid adducts selected from: N_{ε}-fructosyl-lysine (FL), glyoxal-derived hydroimidazolone (G-H1), 3-deoxyglucosone-derived hydroimidazolone (3DG-H), N_{ε}-carboxymethyl-lysine (CML), N_{ω}-carboxymethylarginine (CMA), methylglyoxal-derived hydroimidazolone (MG-H1), pyrraline, methylglyoxal-derived lysine dimer (MOLD), dityrosine (DT), N-formylkynurenine (NFK), α-aminoadipic semialdehyde (AASA), glutamic semialdehyde (GSA) and 3-Nitrotyrosine (3-NT).

In some embodiments, the kit comprises reagents for detecting the concentration of an amino acid in a sample, wherein said amino acid is one or more selected asparagine, glutamate, glutamine, proline, serine, threonine, tryptophan, valine, or a combination thereof. In some embodiments, the kit comprises reagents for detecting the concentration of creatinine in a sample.

In some embodiments, the kit comprises reagents for detecting the concentration of an amino acid adduct and/or amino acid by selected reaction monitoring (SRM) mass spectrometry, Western Blot, Enzyme-Linked Immunosorbent Assay (ELISA), liquid chromatography mass spectrometry (LC-MS), reverse phase mass spectrometry, surface enhanced laser desorption ionization time-of-flight mass spectrometry (SELDI-TOF), matrix assisted laser desorption ionization time-of-flight mass spectrometry (MALDI-TOF), liquid chromatography-tandem mass spectrometry, isotope dilution mass spectrometry, size permeation (gel filtration), ion exchange, affinity, high performance liquid chromatography, ultra performance liquid chromatography, one-dimensional gel electrophoresis (1-DE), and/or two-dimensional gel electrophoresis (2-DE).

In some embodiments, the kit comprises reagents for detecting the concentration of an amino acid adduct by liquid chromatography-tandem mass spectrometry.

In some embodiments, the kit comprises standards for use in detecting the concentration of an amino acid adduct by liquid chromatography-tandem mass spectrometry.

In certain embodiments, the kit comprises instructions for use any of the previous embodiments.

### BRIEF DESCRIPTION OF THE DRAWINGS

FIG. 1A shows the structure of the early glycation adduct FL. FIG. 1B shows the structure of the nitration adduct 3-NT. FIG. 1C shows the structure of the advanced glycation end products CML, CEL, pyrraline, G-H1, MG-H1, 3DG-H, CMA, Glucosepane, and MOLD. FIG. 1D shows the structure of the oxidation adducts DT, NFK, 3-NT, AASA, and GSA.

FIG. 2 shows a scheme of the training and validation subject groups of diagnostic algorithms for detection of autistic spectrum disorder.

FIG. 3 shows demographic and clinical features of the autistic children group. † Onset pattern was defined according to Ozonoff et. al. (Autism Journal: Official journal of the International Society for Autism Research. 2008; 1 (6): 320-8) $ Probability the subject has autism estimated from diagnostic algorithms derived from the experimental biomarker data (algorithm performance is shown in FIG. 14).

FIG. 4 shows glycation, oxidation and nitration adduct residue content of plasma protein. Data are median (lower-upper quartile); healthy controls, n=21, and ASD, n=27. Significance (Mann-Whitney U); *, ** and ***, P<0.05, P<0.01 and P<0.001 after Bonferroni correction of 14 applied.

FIG. 5 shows plasma glycation, oxidation and nitration free adduct content in plasma filtrate. Data are median (lower-upper quartile); healthy controls, n=21-31, and ASD, n=27-38. Significance (Mann-Whitney U); *, P<0.05 after Bonferroni correction of 15 applied.

FIG. 6 shows urinary glycation, oxidation and nitration free adduct content. Data are median (lower-upper quartile); healthy controls, n=21-31, and ASD, n=27-38. Significance (Mann-Whitney U); *, P<0.05 after Bonferroni correction of 15 applied.

FIG. 7 shows plasma amino acid metabolome content. Data are Mean±SD or median (lower-upper quartile); healthy controls, n=21, and ASD, n=27. Significance: t-test for parametric data and Mann-Whitney U for non-parametric data. *, p<0.05, **, P<0.01 after Bonferroni correction of 20 applied.

FIG. 8 shows urinary amino acid metabolome content. Data are Mean±SD or median (lower-upper quartile); healthy controls, n=21, and ASD, n=27. Significance: t-test for parametric data and Mann-Whitney U for non-parametric data. *, p<0.05, **, P<0.01 after Bonferroni correction of 20 applied.

FIG. 9 shows renal clearance values of glycation, oxidation and nitration free adducts. Data are mean±SD or median (lower-upper quartile); healthy controls, n=21, and ASD, n=27. Significance: t-test for parametric data and Mann-Whitney U for non-parametric data. *, p<0.05, after Bonferroni correction of 14 applied. #ml/mg creatinine.

FIG. 10 shows renal clearance values of amino acids. Data are mean±SD or median (lower-upper quartile); healthy controls, n=21, and ASD, n=27. Significance: t-test for parametric data and Mann-Whitney U for non-parametric data. *, p<0.05, after Bonferroni correction of 20 applied.

FIG. 11A provides a heatmap illustrating concentration changes of glycation, oxidation and nitration adducts and amino acid metabolome in plasma and urine. FIG. 11B A heatmap showing changes of the amino acid metabolome in plasma and urine. The key (Δlog2) shown in FIG. 11A applies likewise to FIG. 11B.

FIG. 12 shows data distributions of biomarkers with significantly different changes in the ASD study group after Bonferroni correction. FIG. 12A Protein adduct residues CML, CMA, and DT. FIG. 12B Plasma free adduct CMA. FIG. 12C Urine free adducts DT and GSA; and amino acids Asn, Pro, Ser, and Val. FIG. 12D Renal clearance of CMA and Arg. Significance: one asterisk, two asterisks, and three asterisks indicate P<0.05, P<0.01, and P<0.001, respectively.

FIG. 13 shows receiver operating characteristic plots of diagnostic algorithms for detection of autistic spectrum disorder by protein glycation and oxidation adducts. FIG. 13A Algorithm-1, plasma protein adduct residues. AUROC=0.96. FIG. 13B Algorithm-2, plasma free amino acid adducts. AUROC=0.78. FIG. 13C Algorithm-3, plasma protein adduct residues and free adducts. AUROC=0.99. FIG. 13D Algorithm-4, urine free amino acid adducts. AUROC=0.78. ROC plots are representative results from one run of the classification experiment. A random outcome is AUROC=0.50.

FIG. 14 shows details of diagnostic algorithms developed for autistic spectrum disorder from plasma and urinary analytes. Algorithm outcomes for 2-fold cross-validation (10 randomized repeat trials for robustness) using SVMs (95% CI given in brackets).

FIG. 15 shows a schematic explanation for changes found in protein damage and amino acids in ASD. FIG. 15A Proposed mechanism for observed changes found in plasma protein glycation and oxidation adducts. FIG. 15B Transport of Arg and CMA across the renal tubular epithelium and proposed mechanism for increased renal CL (increased Arg and CMA reuptake). Key: grey filled arrows show processes; black-filled arrows show changes observed FIG. 15A and changes expected FIG. 15B in ASD.

FIG. 16 shows mass spectrometric multiple reaction monitoring detection of protein glycation, oxidation and nitration adducts and amino acids.

FIG. 17 shows correlation analysis of plasma protein glycation, oxidation and nitration adduct residues. Correlation coefficients; Spearman (P<0.01).

FIG. 18 shows correlation analysis of plasma protein glycation, oxidation and nitration free adducts. Correlation coefficients; Spearman (P<0.01).

FIG. 19 shows correlation analysis of plasma amino acids. Correlation coefficients; Spearman (P<0.01).

FIG. 20 shows correlation analysis of urinary protein glycation, oxidation and nitration free adducts. Correlation coefficients; Spearman (P<0.01).

FIG. 21 shows correlation analysis of urinary amino acids. Correlation coefficients; Spearman (P<0.01).

FIG. 22 shows a confusion matrix of algorithms to identify autistic spectrum disorder. The confusion matrices shown are representative results from one run of the classification experiment.

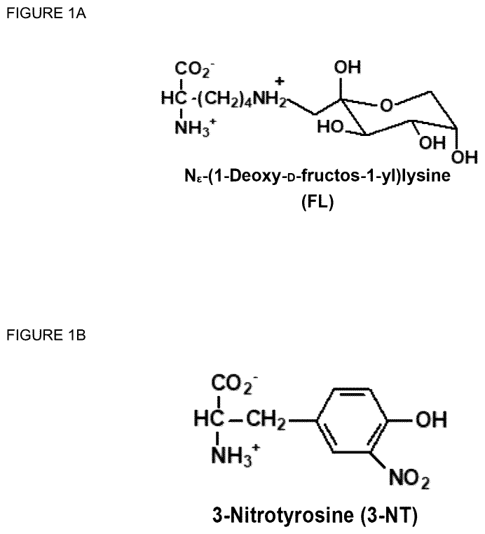

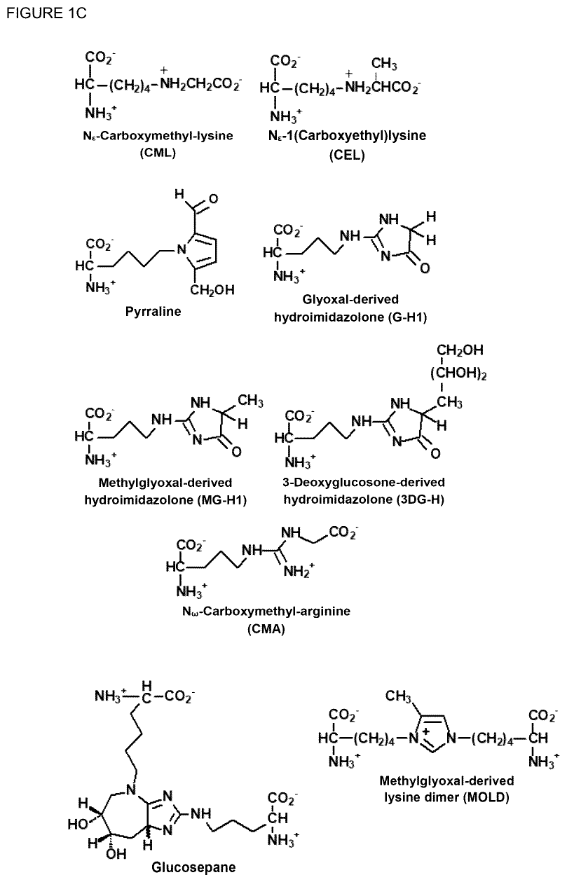

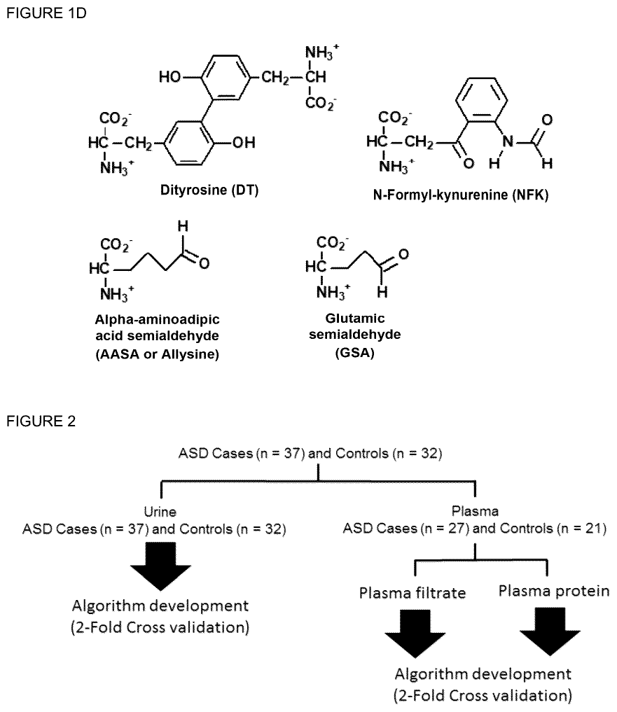

### DETAILED DESCRIPTION

### Definitions

As used herein, the term “Autistic Spectrum Disorder” or “Autism Spectrum Disorder” (“ASD”) refers to a diverse group of developmental disorders typically affecting social interactions and range of interests and causing a wide spectrum of other disabilities, such as speech disturbances, repetitive and/or compulsive behaviors, hyperactivity, anxiety and difficulty to adapt to new environments, with or without cognitive impairment. In some embodiments, the such disorders may include autism, Asperger syndrome, pervasive developmental disorder not otherwise specified (PDD-NOS) and childhood disintegrative disorder. In some embodiments, the term ASD is autism.

As used herein, the terms “subject”, “individual” and “patient” are used interchangeably herein to refer to a mammalian subject. In one embodiment the “subject” is a human, a companion animal (e.g. a pet such as dogs, cats, and rabbits), livestock (e.g. pigs, sheep, cattle, and goats), and horses. In a preferable embodiment, the subject is a human, such as a human child. In methods of the invention, the subject may not have been previously diagnosed as having ASD. Alternatively, the subject may have been previously diagnosed as having ASD. The subject may also be one who exhibits disease risk factors, or one who is asymptomatic for ASD. The subject may also be one who is suffering from or is at risk of developing ASD. In one embodiment, the subject is a typically developing subject. A person of skill would understand that a typically developing subject is a subject at high risk of having or developing ASD. Thus, in one embodiment, the methods described herein may be used to confirm the presence of ASD in a subject. For example, the subject may previously have been diagnosed with ASD through analysis of symptoms that the subject presents.

As used herein, the term “concentration” refers the total concentration of the amino acid adduct that is detected in a sample.

As used herein, the term “relative” or “relative concentration” refers to the difference between the amino acid adduct detected in a sample and e.g. another constituent of the sample.

As used herein, the term “blood sample” refers to a whole blood as well as a sample obtained after subjecting blood to one or more processing steps. In certain embodiments, the processing steps comprise fractionation. In certain embodiments the blood may be a blood plasma or blood serum sample. In certain embodiments, the sample may be a blood sample containing proteins. In certain embodiments, the blood sample excludes amino acids. In certain embodiments, the blood sample excludes proteins. In one embodiment, the sample is a blood plasma sample. In a certain embodiment, the blood sample comprises a blood plasma sample which comprises amino acid adducts comprised in a polypeptide sequence, and lacks free amino acid adducts.

As used herein, the term “plasma ultrafiltrate” refers to a sample obtained by subjecting blood plasma to ultrafiltration to isolate a free amino acid adduct.

As used herein, the term “urine ultrafiltrate” refers to a sample obtained by subjecting urine to ultrafiltration to isolate a free amino acid adduct.

As used herein, the term “amino acid adduct” refers to an amino acid that has been oxidized, nitrated, or glycated. The amino acid adduct may be comprised in a polypeptide sequence. In certain embodiments, the polypeptide sequence is an “adduct residue”. In certain embodiments, the amino acid adduct is a “free adduct.” In some embodiments, a “free adduct” may be a proteolytic digestion product released into body fluid of a subject following proteolysis of a polypeptide sequence comprising the amino acid adduct. In some embodiments, the concentration of free adducts is detected. In some embodiments, the concentration of amino acid adducts present in a polypeptide sequence are detected.

As used herein, the term “plasma ultrafiltrate” refers to a sample obtained by subjecting blood plasma to ultrafiltration to isolate a free amino acid adduct.

As used herein, the term “urine ultrafiltrate” refers to a sample obtained by subjecting urine to ultrafiltration to isolate a free amino acid adduct.

As used herein, the term “amino acid adduct” refers to an amino acid that has been oxidized, nitrated, or glycated.

Alternatively, or additionally, the concentration of amino acid adducts present in a polypeptide sequence are detected.

As used herein, the term “one or more” refers to when used in the context of an amino acid adduct described herein means at least 1, 2, 3, 4, 5, 6, 7, 8, 9, 10, or 11, of the amino acid adducts. In some embodiments, the term “one or more” when used in the context of an amino acid adduct described herein means at least 1, 2, 3, 4, 5, 6, 7, 8, 9, 10, 11, 12, 13, 14, or 15 of the amino acid adducts. In certain embodiments, In a preferred the term “one or more” refers to when used in the context of an amino acid adduct described herein means at least 2 of the amino acid adducts. In certain embodiments, the term “one or more” refers to when used in the context of an amino acid adduct described herein means all of the amino acid adducts.

A “non-ASD reference standard” refers a sample obtained from a subject that does not have ASD. In some embodiments, a “non-ASD reference standard” may be obtained from a subject who has not been diagnosed with ASD and does not exhibit a symptom of ASD.

As used herein, the term “ASD reference standard” refers to a sample obtained from a subject that has ASD. In some embodiments, an “ASD reference standard” may be obtained from a subject who has been diagnosed with ASD and exhibits one or more symptoms of ASD.

As used herein, the term “disease status” may be used synonymously with “ASD status.” In certain embodiments, the presence of ASD corresponds to an unhealthy status. In certain embodiments, the lack of ASD correspond to a healthy status.

As used herein, the phrase “classifying the health of the subject based on the concentration the amino acid detected in the sample with a diagnostic algorithm” refers to the statistical or machine learning classification process by which the concentration of the amino acid adduct in the test sample is used to determine a category of health with a diagnostic algorithm, typically a statistical or machine learning classification algorithm.

As used herein, the term “classifying the health of the subject” refers to classifying the subject as having ASD, or alternatively classifying the subject as not having ASD.

As used herein, the phrase “training the diagnostic algorithm” refers to the supervised learning of a diagnostic algorithm on the basis of concentrations for each amino acid adduct obtained from a population of subjects having known ASD health.

As used herein, the phrase “training the diagnostic algorithm” refers to variable selection in a statistical model on the basis of concentrations for each amino acid adduct obtained from a population of subjects having known ASD health. In some embodiments, training a diagnostic algorithm may for example include determining a weighting vector in feature space for each category, or determining a function or function parameters.

As used herein, the term “population of subjects” means one or more subjects. In one embodiment, the population of subjects consists of one subject. In one embodiment, the population of subjects comprises multiple subjects. As used herein, the term “multiple” refers to at least 2 (such as at least 4, 6, 8, 10, 12, 14, 16, 18, 20, 22, 24, 26, 28, or 30) subjects. In one embodiment, the population of subjects comprises at least 30 subjects.

As used herein, the term “statistically deviates” means that the concentrations of the amino acid adducts detected for the test subject differs from those detected for the reference population to a statistically significant level. In some embodiments, the deviation in marker abundance may be an increase or decrease.

As used herein, the term “sensitivity” refers to the percentage of subjects who were correctly identified as having ASD. In some embodiments, the term “sensitivity” is defined in the art as the number of true positives divided by the sum of true positives and false negatives. In some embodiments, the sensitivity of the methods of the invention may be at least about 65%, 70%, 75%, 77%, 80%, 85%, 86%, 87%, 88%, 89%, 90%, or 92%.

As used herein, the term “specificity” refers to the percentage of patients who were correctly identified as not having ASD. In some embodiments, the term “specificity” refers to the number of true negatives divided by the sum of true negatives and false positives. In some embodiments, the specificity may be at least about 67%, 70%, 75%, 80%, 84%, 86%, 87%, 88%, 89%, or 90%.

As used herein, the term “earlier timepoint” refers to a timepoint of at least 24 hours, at least 48 hours, at least 72 hours, at least 96 hours, at least 120 hours, at least 1 week, at least 2 weeks, at least 3 weeks, at least 4 weeks, at least 8 weeks, at least 12 weeks, at least 16 weeks, at least 20 weeks, at least 30 weeks, at least 40 weeks, at least 52 weeks, at least 2 years, at least 3 years, or at least 4 years earlier.

As used herein, the term “amino acids” refers to the name of the amino acid, the three letter abbreviation, or the single letter abbreviation.

As used herein, the term “protein,” refers to proteins, polypeptides, and peptides. As used herein, the term “amino acid sequence” refers to the term “polypeptide” and/or the term “protein.” In some embodiments, the term “amino acid sequence” is synonymous with the term “peptide.” In some embodiments, the term “amino acid sequence” is synonymous with the term “enzyme”. In some embodiments, the terms “protein” and “polypeptide” are used interchangeably herein. In certain embodiments, the conventional one-letter and three-letter codes for amino acid residues may be used. In certain embodiments, the 3-letter code for amino acids as defined is in conformity with the IUPACIUB Joint Commission on Biochemical Nomenclature (JCBN). In certain embodiments, a polypeptide may be coded for by more than one nucleotide sequence due to the degeneracy of the genetic code.

As used herein, the following abbreviations are used: AASA, α-aminoadipic semialdehyde; ADOS, autism diagnostic observation schedule; AGE, advanced glycation end product; ASD, Autism spectrum disorder; AUROC, area under-the-curve of receiver operating characteristic plot; b0,+AT, solute carrier 7, member 9; CARS, childhood autism rating scale; CAT-3, cationic amino acid transporter-3; CD98hs, cluster of differentiation heavy subunit; CEL, N_{ε}-(1-carboxyethyl) lysine; CL, renal clearance; CMA, N_{ω}-carboxymethylarginine; CML, N_{ε}-carboxymethyl-lysine; 3-DG, 3-deoxyglucosone; 3DG-H, hydroimidazolone AGEs derived from 3-deoxyglucosone; DT, dityrosine; DUOX, dual oxidase; EDTA, ethylenediaminetetra-acetic acid; FL, N_{ε}-fructosyl-lysine; G-H1, hydroimidazolone AGE derived from glyoxal; GSA, glutamic semialdehyde; GSP, glucosepane; hLAT-1, large neutral amino acid transporter subunit-1; LC-MS/MS, liquid chromatography-tandem mass spectrometry; Leiter-R, Leiter international performance scale-revised; MG, methylglyoxal; MG-H1, hydroimidazolone AGE derived from methylglyoxal MOLD, methylglyoxal-derived lysine crosslink; MRM, multiple reaction monitoring; NFK, N-formylkynurenine; 3-NT, 3-nitrotyrosine; PEP-3, psychoeducational profile-3; rBAT, neutral and basic amino acid transport protein; ROS, reactive oxygen species; SLC7A5, solute carrier family 7, member 5; SVM, support vector machines; TD, Typically developing; UPR, unfolded protein response; y+LAT-1, solute carrier family 7 member 7; y+LAT-2, solute carrier family 7 member 6.

As used herein, the term “statistically similar” refers to the concentrations of the amino acid adducts detected for the test subject are similar to those detected for the reference population to a statistically significant level.

As used herein, the term “statistically significant” refers that the alteration is greater than what might be expected to happen by chance alone (e.g. p=<0.05). Statistical significance can be determined by any method known in the art.

As used herein, the term “healthy subject” refers to an individual subject or group of subjects who have not shown any symptoms of ASD, have not been diagnosed with ASD and/or are not likely to develop ASD. Preferably said healthy subject(s) is not on medication affecting the disease and has not been diagnosed with any other disease. The one or more healthy subjects may have a similar sex, age and body mass index (BMI) as compared with the test subject.

As used herein, where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein the terms “a,” “an,” and “the” include plural referents unless the context clearly dictates otherwise. Thus, for example, reference to “an amino acid adduct” includes a plurality of such amino acid adducts and reference to “the amino acid adduct” includes reference to one or more amino acid adducts and equivalents thereof known to those skilled in the art, and so forth.

Unless defined otherwise, as used herein, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

SAMPLE

In some embodiments, the sample comprises an amino acid adduct.

In some embodiments, the sample is isolated from a subject suspected of having ASD. In some embodiments, the sample is isolated from a subject diagnosed as having ASD.

In some embodiments, the sample is selected from one or more of blood, urine, eye fluid, lymphatic fluid, saliva, synovial fluid, seminal fluid, cerebrospinal fluid, sebaceous secretions, and/or sputum, or any combination thereof. In some embodiments, the sample is pre-treated for analysis by conventional techniques as described herein and known by those skilled in the art. In a certain embodiment, the sample is hydrolyzed prior to detection.

In a certain embodiment, the hydrolysis is performed enzymatically. In a certain embodiment, the enzymatic digestion comprises treatment with pepsin. In certain embodiments, the enzyme digestion further comprises digestion with pronase E, prolidase, and aminopeptidase. In a certain embodiment, the enzyme digestion further comprises collagenase. In a certain embodiment, the enzymatic digestion comprises automated exhaustive enzymatic hydrolysis.

In a certain embodiment, prior to hydrolysis, the proteins are washed by ultrafiltration to remove free amino acids, and retained protein is collected for hydrolysis. In a certain embodiment, the retained protein may be delipidated prior to hydrolysis.

In some embodiments, the sample is a blood sample.

In some embodiments, the sample is processed to isolate a free amino acid adduct from a sample by ultrafiltration prior to detecting the concentration of amino acid adducts in a sample.

In some embodiments, the method comprises collecting the oxidized, nitrated, and glycated free adducts are collected by microspin ultrafiltration. In a certain embodiment, the ultrafiltration step comprises a molecular weight cut-off of at least about 10 kDa. In a certain embodiment, the molecular weight cut-off may be at least about 5 kDa, 6 kDa, 7 kDa, 8 kDa, 9 kDa, 10 kDa, 11 kDa, 12 kDa, 13 kDa, 14 kDa, or 15 kDa. In certain embodiments, the ultrafiltration step is performed at a temperature of between about 2° C. and 10° C., such as at about 4° C.

### Amino Acid Adducts

In some embodiments, the method comprises detecting an amino acid adduct or any isomer thereof. In a certain embodiment, the method comprises detecting 3DG-H. In a certain embodiment, the method comprises detecting 3DG-H in one or more structural isomers. In a certain embodiment, the method comprises detecting MG-H1 in one or more structural isomers. In a certain embodiment, the method comprises detecting all the structural isomers of MG-H1 and G-H1. In a certain embodiment, the method comprises detecting an isomer of MG-H1 and an isomer of G-H1.

In some embodiments, the amino acid adduct is a glycated amino acid adduct.

In some embodiments, the amino acid adduct is an oxidized amino acid adduct.

In some embodiments, the amino acid adduct is selected from dityrosine (DT), N-formylkynurenine (NFK), α-aminoadipic semialdehyde (AASA), glutamic semialdehyde (GSA), or any combination thereof.

In some embodiments, the amino acid adduct is a nitrated amino acid adduct. In a certain embodiment, the nitrated amino acid adduct is 3-Nitrotyrosine (3-NT).

In some embodiments, the methods comprise detecting the concentration of a glycated amino acid, an oxidized amino acid, and/or a nitrated amino acid. In certain embodiments, the method comprises detecting the concentration of a glycated amino acid and an oxidized amino acid.

In some embodiments, the concentration of a conventional amino acid may also be detected. In certain embodiments, the conventional amino acids are selected from: alanine, cysteine, aspartate, glutamate, phenylalanine, glycine, histidine, isoleucine, lysine, leucine, methionine, asparagine, pyrrolysine, proline, glutamine, arginine, serine, threonine, selenocysteine, valine, tryptophan, tyrosine, or any combination thereof.

In some embodiments, a glycated amino acid adduct is selected from N_{ε}-fructosyl-lysine (FL), glyoxal-derived hydroimidazolone (G-H1), N_{ε}-(1-carboxyethyl) lysine (CEL), 3-deoxyglucosone-derived hydroimidazolone (3DG-H), N_{ε}-carboxymethyl-lysine (CML), N_{ω}-carboxymethylarginine (CMA), methylglyoxal-derived hydroimidazolone (MG-H1), pyrraline, glucosepane (GSP), and methylglyoxal-derived lysine dimer (MOLD), or any combination thereof.

In some embodiments, the amino acid adduct is one or more adducts selected from N_{ε}-carboxymethyl-lysine (CML), 3-deoxyglucosone-derived hydroimidazolone (3DG-H), N_{ω}-carboxymethylarginine (CMA), dityrosine (DT), glutamic semialdehyde (GSA), glyoxal-derived hydroimidazolone (G-H1), pyrraline, methylglyoxal-derived hydroimidazolone (MG-H1), N_{ε}-fructosyl-lysine (FL), N_{ε}-(1-carboxyethyl) lysine (CEL), α-aminoadipic semialdehyde (AASA), and methylglyoxal-derived lysine dimer (MOLD). In a certain embodiment, the amino acid adduct is selected from CML, 3DG-H, CMA, GSA, G-H1, pyrraline, MG-H1, FL, CEL, AASA, and MOLD, or any combination thereof.

In some embodiments, the amino acid adduct is selected from N_{ε}-carboxymethyl-lysine (CML), 3-deoxyglucosone-derived hydroimidazolone (3DG-H), N_{ω}-carboxymethylarginine (CMA), dityrosine (DT), glutamic semialdehyde (GSA), glyoxal-derived hydroimidazolone (G-H1), and pyrraline, or any combination thereof. In a certain embodiment, the amino acid adduct is one or more selected from CML, 3DG-H, GSA, G-H1, and pyrraline, or any combination thereof.

In some embodiments, the amino acid adduct is one or more selected from Nε-fructosyl-lysine (FL), glyoxal-derived hydroimidazolone (G-H1), 3-deoxyglucosone-derived hydroimidazolone (3DG-H), N_{ε}-carboxymethyl-lysine (CML), N_{ω}-carboxymethylarginine (CMA), methylglyoxal-derived hydroimidazolone (MG-H1), pyrraline and/or methylglyoxal-derived lysine dimer (MOLD), dityrosine (DT), N-formylkynurenine (NFK), α-aminoadipic semialdehyde (AASA), and glutamic semialdehyde (GSA), or any combination thereof.

In some embodiments, the amino acid adduct is one or more selected from 3-deoxyglucosone-derived hydroimidazolone (3DG-H), N_{ε}-carboxymethyl-lysine (CML), No-carboxymethylarginine (CMA), dityrosine (DT), and glutamic semialdehyde (GSA), or any combination thereof.

In some embodiments, the amino acid adduct is one or more selected from glyoxal-derived hydroimidazolone (G-H1), 3-deoxyglucosone-derived hydroimidazolone (3DG-H), N_{ε}-carboxymethyl-lysine (CML), N_{ω}-carboxymethylarginine (CMA), pyrraline, dityrosine (DT), α-aminoadipic semialdehyde (AASA) and glutamic semialdehyde (GSA).

In some embodiments, the amino acid adduct is one or more selected from N_{ε}-carboxymethyl-lysine (CML), N_{ω}-carboxymethylarginine (CMA), 3-deoxyglucosone-derived hydroimidazolone (3DG-H), and dityrosine (DT).

In some embodiments, the amino acid adduct is one or more selected from N_{ε}-carboxymethyl-lysine (CML) and N_{ω}-carboxymethylarginine (CMA).

In some embodiments, the amino acid adduct is one or more selected from N_{ε}-carboxymethyl-lysine (CML), N_{ω}-carboxymethylarginine (CMA), 3-deoxyglucosone-derived hydroimidazolone (3DG-H), glyoxal-derived hydroimidazolone (G-H1), dityrosine (DT), and glutamic semialdehyde (GSA).

In a certain embodiment, the amino acid adduct is one or more pyrraline and glutamic semialdehyde (GSA).

### Correlation of Amino Acid Adducts and Disease

In some embodiments, the methods comprise detecting any of the amino acid adducts in any of the previous embodiments. In certain embodiments, the presence of the amino acid adducts in any of the previous embodiments indicate the presence of ASD. In some embodiments, a lack of amino acids adducts in any of the previous embodiments indicate the absence of ASD.

In some embodiments, the method comprises detecting whether the amino acid adducts, in any of the previous embodiments, in a sample is less than or equal to the concentration of amino acid adducts in a non-ASD reference standard. In certain embodiments, ASD is not diagnosed if the concentration of amino acid adducts, in any of the previous embodiments, in a sample is less than or equal to the amino acid adducts in a non-ASD reference standard. In certain embodiments, ASD is diagnosed if the concentration of amino acid adducts, in any of the previous embodiments, in a sample is less than or equal to the amino acid adducts in a non-ASD reference standard.

In some embodiments, the method comprises detecting whether the amino acid adducts, in any of the previous embodiments, in a sample is greater than or equal to the concentration of amino acid adducts in a non-ASD reference standard. In certain embodiments, ASD is diagnosed if the concentration of amino acid adducts, in any of the previous embodiments, in a sample is greater than or equal to the amino acid adducts in a non-ASD reference standard. In certain embodiments, ASD is not diagnosed if the concentration of amino acid adducts, in any of the previous embodiments, in a sample is greater than or equal to the amino acid adducts in a non-ASD reference standard.

### Renal Clearance

In some embodiments, the methods comprise analyzing the renal clearance of amino acid adducts. In some embodiments, the method comprises measuring the renal clearance of an amino acid adduct. In certain embodiments, the renal clearance of amino acid adducts is deduced from the concentration of an amino acid adduct detected in a plasma and urine sample according to the following equation: CL (μl/mg creatinine or ml/mg creatinine)=[Analyte]_{Urine} (nmol/mg creatinine)/[Analyte]_{Plasma} (pmol/ml or nmol/ml).

In some embodiments, the renal clearance of amino acid adducts from any of the previous embodiments is measured.

### Reference Standards

In some embodiments, the method comprises a reference standard. In certain embodiments, the reference standard is an ASD reference standard, a non-ASD reference standard, or a combination thereof.

In some embodiments, the reference standard is age and/or gender matched to the subject from which the sample to be used in a method is obtained.

In some embodiments, the reference standard is analyzed to determine the concentration of an amino acid adduct before, during, or after, the analysis of the amino acid adduct in the sample.

In certain embodiments, the method detects a concentration difference between the sample and the reference standard. In certain embodiments, the concentration difference indicates the presence of ASD. In certain embodiments, the method detects no concentration difference between the sample and the reference standard. In certain embodiments, the absence of a concentration difference indicates the absence of ASD.

In some embodiments, the method correlates that an increase in the concentration of an amino acid adduct, when compared to a non-ASD reference standard, with the presence of ASD. In certain embodiments, the method correlates that a decrease in an amino acid adduct concentration, when compared to the non-ASD reference standard, is not indicative of the presence of ASD. In certain embodiments, the method correlates that no change in the concentration of an amino acid adduct, when compared to the non-ASD reference standard, is not indicative of the presence of ASD.

In some embodiments, the method correlates that a decrease in the concentration of an amino acid adduct, when compared to a non-ASD reference standard, with the presence of ASD. In certain embodiments, an increase in the concentration of an amino acid adduct, when compared to the non-ASD reference standard, is not indicative of the presence of ASD. In certain embodiments, no change in the concentration of an amino acid adduct, when compared to the non-ASD reference standard, is not indicative of the presence of ASD.

### Diagnostic Algorithm

In some embodiments, the methods comprise use of a diagnostic algorithm or other data-driven combinatorial approach. In some embodiments, the methods comprise detecting the concentrations of amino acid adducts and entering the values into a diagnostic algorithm. In a certain embodiment, the algorithm indicates whether ASD is present or absent.

In some embodiments, a diagnostic algorithm is one or more selected from: Random Forests, logistic regression, ensemble classifier, Support Vector Machines (SVMs), general linear models (GLM), and GLMNET. In a certain embodiment, the algorithm is an SVM.

In some embodiments, the methods comprise using a diagnostic algorithm configured to diagnose the presence or absence of ASD based on the concentration of the amino acid adduct detected in the sample, wherein the diagnostic algorithm is trained on the corresponding concentration for the same amino acid adduct in one or more reference standards. In some embodiments the algorithm provides different predictive weightings to different amino acid adducts to improve specificity and/or sensitivity and/or accuracy of the diagnostic method.

In some embodiments, any of the previous embodiments, may be used in methods for determining prognosis, identifying a therapy suitable for treating ASD, and monitoring the efficacy of an ASD therapy are also provided.

In some embodiments, the method comprises identifying a therapeutic as an ASD therapy. In some embodiments, the method comprises monitoring drug efficacy of an ASD therapy. In certain embodiments, an effective drug is determined by the absence of ASD. In certain embodiments, a non-effective drug is determined by the lack of ASD.

In some embodiments, the methods comprise using a diagnostic algorithm configured to classify the health of the subject based on the concentration of the amino acid adduct detected in the sample with said diagnostic algorithm. In certain embodiments, the diagnostic algorithm is trained on the corresponding concentration for the same amino acid adduct obtained from a population of subjects having known disease status.

In some embodiments, a diagnostic algorithm is developed using a machine learning approach. In certain embodiments, the diagnostic algorithm is trained by two fold cross-validation. In certain embodiments, the two fold cross-validation is trained based on the concentration of an amino acid adduct detected in 50% of the ASD and non-ASD (control) subjects (training subset) before being used to predict the disease status for the remaining 50% of subjects (test set).

In some embodiments, a 2-class diagnostic algorithm developed via a machine learning approach. In certain embodiments, the 2-class diagnostic algorithm is trained based on the concentration of an amino acid adduct in a sample from a known ASD subject and known non-ASD subject, before being used to classify the health of a test subject. In certain embodiments, the classification by a diagnostic algorithm includes a scoring likelihood of a panel of amino acid adduct concentrations belonging to one or more categories, and determining the highest-scoring category. In certain embodiments, the diagnostic algorithm classifies a panel of amino acid adduct concentrations to previous observations by means of a distance function.

In some embodiments, diagnostic algorithms suitable for classification include, but are not limited to support vector machines, ensemble classifier algorithms, random forests, logistic regression, or any combination thereof. In certain embodiments, the logistic regression comprises multiclass or multinomial logistic regression, and/or algorithms adapted for sparse logistic regression.

In some embodiments, the diagnostic algorithm is trained either internally, externally, or any combination thereof. In a certain embodiment, the method comprises a step of training of a diagnostic algorithm. In a certain embodiment, the diagnostic algorithm is trained externally to the method of the invention and accessed during the classification step of the invention.

In some embodiments, the diagnostic algorithm is trained by detecting the concentration of an amino acid adduct in a sample obtained from a population of healthy subject(s).

In some embodiments, the diagnostic algorithm is trained by detecting the concentration of an amino acid adduct in a sample obtained from a population of subjects suffering from ASD.

In some embodiments, an amino acid adduct concentration profile characteristic of ASD is determined to provide a reference profile. In certain embodiments, the profile of concentrations from a sample are compared to the reference profile to determine whether the test subject also has ASD. In some embodiments, a diagnostic algorithm trained to classify ASD, is used to classify whether the test subject also has ASD.

In some embodiments, the population of subjects used to obtain reference standards for the diagnostic algorithm, and/or the population of subjects used to train the diagnostic algorithm, comprise: at least one (healthy) subject having no ASD, and/or at least one subject having ASD. In a certain embodiment, the population of subjects may comprise: one or more subjects having no ASD, and/or one or more subjects having ASD.

In some embodiments, the methods comprise comparing the concentration of an amino acid adduct in a sample obtained from a test subject to the concentration of an amino acid adduct for a reference standard obtained from a population of subjects having known ASD health, to determine the ASD health of the subject.

In some embodiments, the method comprises classifying the health of a subject based on the concentration of an amino acid adduct with a diagnostic algorithm trained on corresponding concentration obtained from a population of subjects having known health, to determine whether a subject has or does not have ASD. In certain embodiments, the method comprises classifying a subject as belonging to or not belonging to the reference population. In certain embodiments, the classification comprises determining whether the concentration of amino acid adducts in the subject are statistically similar to the reference population or statistically deviate from the reference population.

In some embodiments, the method comprises diagnosing a subject as having ASD when the concentration of amino acid adducts detected is statistically similar to the concentration determined for the corresponding amino acid adduct concentrations detected in a population of subjects having ASD. In some certain embodiments, the method comprises diagnosing a subject as having no ASD when the concentration of amino acid adducts detected is statistically similar to the amount determined for the corresponding values obtained from a population of subjects having no ASD.

In some embodiments, the method comprises diagnosing a subject as having ASD when the concentrations of the amino acid adducts detected statistically deviates from the amount determined for the corresponding values obtained from a population of subjects having no ASD. In a certain embodiment, the method comprises diagnosing a subject as having no ASD when the concentration of the amino acid adduct detected statistically deviates from the concentration determined for the corresponding amino acid adduct obtained from a population of subjects having ASD.

In some embodiments, the method comprises classifying a subject using a diagnostic algorithm. In certain embodiments, the diagnostic algorithm is a classification algorithm. In certain embodiments, the classification algorithm is a support vector machine algorithm. In certain embodiments, the classification algorithm is an ensemble algorithm comprising different types of classification algorithms. In a certain embodiment, the classification algorithm is a decision tree based algorithm. In a certain embodiment, the method comprises using regression algorithms. In a certain embodiment, the method comprises using a neural networks. In a certain embodiment, the classification algorithm is a random forest algorithm.

In some embodiments, the method comprises using a computational model based on a diagnostic algorithm adapted to classify the health of a subject based on the concentration of an amino acid adduct detected in the sample with a diagnostic algorithm, wherein the diagnostic algorithm is trained on the corresponding concentration for the amino acid adduct obtained from a population of subjects having known disease status.

In a certain embodiment, the machine learning approach uses a training set comprising an amino acid adduct detected in 50% of the ASD and non-ASD (control) subjects (training subset) before being used to predict the disease status for the remaining 50% of subjects (test set).

In some embodiments, the method comprises an algorithm trained to produce a mathematical equation to determine the probability of a subject having ASD. In some embodiments, the algorithm is trained from estimates of plasma protein contents of modified amino acid residues and/or modified amino acids obtained in implementation of the blood test. In a certain embodiment, the probability is determined by analyzing a plasma sample and using the following equation:

$$Probability of ASD ( \% ) = {k}_{1} \times {{[ {CML}_{res} ]}^{y}}_{1} + {k}_{2} \times {{[ {CMA}_{res} ]}^{y}}_{2} + {k}_{3} \times {{[ 3 DG - {H}_{res} ]}^{y}}_{3} + {k}_{4} \times {{[ {DT}_{res} ]}^{y}}_{4}$$

where: [CMLᵣₑₛ], [CMAᵣₑₛ], [3DG-Hᵣₑₛ] and [DTᵣₑₛ] are N_{ε}-carboxymethyl-lysine (CML), N_{ω}-carboxymethyl-arginine (CMA), 3-deoxyglucosone-derived hydroimidazolone (3DG-H) and dityrosine (DT) residue contents of plasma protein in units of mmol/mol lysine, mmol/mol arginine, mmol/mol arginine and mmol/mol tyrosine, respectively, determined in test samples by the enzymatic hydrolysis-LC-MS/MS analysis procedure; and k₁, k₂, k₃ and k₄ are multiplier constants and y₁, y₂, y₃ and y₄ exponents determined empirically in algorithm training.

In a certain embodiment, the probability is determined by analyzing a plasma sample and using the following equation:

$$Probability of ASD ( \% ) = {k}_{1} \times {{[ {CML}_{res} ]}^{y}}_{1} + {k}_{2} \times {{[ {CMA}_{res} ]}^{y}}_{2} + {k}_{3} \times {{[ 3 DG - {H}_{res} ]}^{y}}_{3} + {k}_{4} \times {{[ {DT}_{res} ]}^{y}}_{4} + {k}_{5} \times {{( subject age / years )}^{y}}_{5}$$

where k₅ and y₅ is the multiplier constant and exponent determined empirically in algorithm training for an additional term of subject age. Multipliers and exponents have different values depending on whether the subject tested is biologically male or female.

In a certain embodiment, the probability is determined by analyzing plasma ultrafiltrate sample and using the following equation:

$$Probability of ASD ( \% ) = {k}_{1} \times {{[ {CML}_{aa} ]}^{y}}_{1} + {k}_{2} \times {{[ {CMA}_{aa} ]}^{y}}_{2}$$

where: [CMLₐₐ] and [CMAₐₐ] are plasma concentrations of CML and CMA amino acids in units of μM, determined in test samples by the LC-MS/MS analysis procedure; and k₁ and k₂ are multiplier constants and y₁ and y₂ exponents determined empirically in algorithm training.

In a certain embodiment, the probability is determined by analyzing plasma protein and plasma ultrafiltrate sample and using the following equation:

$$Probability of ASD ( \% ) = {k}_{1} \times {{[ {CML}_{res} ]}^{y}}_{1} + {k}_{2} \times {{[ {CMA}_{res} ]}^{y}}_{2} + {k}_{3} \times {{[ 3 DG - {H}_{res} ]}^{y}}_{3} + {k}_{4} \times {{[ {DT}_{res} ]}^{y}}_{4} + {k}_{5} \times {{[ G - H {1}_{aa} ]}^{y}}_{5} + {k}_{6} \times {{[ {GSA}_{aa} ]}^{y}}_{6}$$

where: [CMLᵣₑₛ], [CMAᵣₑₛ], [3DG-Hᵣₑₛ] and [DTᵣₑₛ] are CML, CMA, 3DG-H and DT residue contents of plasma protein in units of mmol/mol lysine, mmol/mol arginine, mmol/mol arginine and mmol/mol tyrosine, respectively, determined in test samples by the enzymatic hydrolysis-LC-MS/MS analysis procedure and [G-H1ₐₐ] and [GSAₐₐ] are plasma concentrations of glyoxal-derived hydroimidazolone (G-H1) and glutamic semialdehyde (GSA) amino acids determined in test samples by the LC-MS/MS analysis procedure; and k₁, k₂, k₃, k₄, k₅ and k₆ are multiplier constants and y₁, y₂, y₃, y₄, y₅ and y₆ exponents determined empirically in algorithm training.

In a certain embodiment, the probability is determined by analyzing a urine sample and using the following equation:

$$Probability of ASD ( \% ) = {k}_{1} \times {{[ {GSA}_{aa} ]}^{y}}_{1} + {k}_{2} \times {{[ {Pyrraline}_{aa} ]}^{y}}_{2}$$

where: [_{GSAaa}] and [Pyraalineₐₐ] are plasma concentrations of GSA and pyrraline amino acids in units of nmol/mg creatinine, determined in test samples by the LC-MS/MS analysis procedure; and k₁ and k₂ are multiplier constants and y₁ and y₂ exponents determined empirically in algorithm training.

In some embodiments, the algorithm training is trained using an autism case and control data with testing by cross-validation. In a certain embodiment, the cross-validation comprises a 5-fold cross validation by training on 80% data selected randomly and tested on the remaining 20% and repeating the process 10 times. In certain embodiments, the algorithms are used to calculate a range of probabilities. In certain embodiments, the range of probability is from 50 to 100% of a subject having autism.

In some embodiments, the algorithms assess the severity of ASD. In a certain embodiment, the severity of ASD is calculated using one or more of the following equations:

$$ADOS - 2 score = {k}_{1} \times {{[ MG - H {1}_{res} ]}^{y}}_{1} ;$$

$$ADOS - 2 score = {k}_{2} \times {{[ {CEL}_{res} ]}^{y}}_{2} ;$$

$$ADOS - 2 score = {k}_{1} \times {{[ MG - H {1}_{res} ]}^{y}}_{1} + {k}_{2} \times {{[ {CEL}_{res} ]}^{y}}_{2} ;$$

where [MG-H1ᵣₑₛ] and [CELᵣₑₛ] are MG-H1 and CEL residue contents of plasma protein in units of mmol/mol arginine and mmol/mol lysine, respectively, determined in test samples by the enzymatic hydrolysis-LC-MS/MS analysis procedure; and k₁ and k₂ are multiplier constants and y₁ and y₂ exponents are determined empirically in algorithm training.

### Kit

In some embodiments, a kit is described comprising reagents for detecting the concentration of an amino acid adduct in a sample, wherein said amino acid adduct is one or more selected from a glycated amino acid adduct, an oxidized amino acid adduct, a nitrated amino acid adduct or a combination thereof, and instructions for use.

In some embodiments, the kit comprises reagents for detecting the concentration of creatinine in a sample.

In some embodiments, the kit comprises reagents for detecting the concentration of an amino acid adduct by a method selected from stable isotopic dilution analysis liquid chromatography-tandem mass spectrometry, reaction monitoring (SRM) mass spectrometry, Western Blot, Enzyme-Linked Immunosorbent Assay (ELISA), liquid chromatography mass spectrometry (LC-MS), reverse phase mass spectrometry, surface enhanced laser desorption ionization time-of-flight mass spectrometry (SELDI-TOF), matrix assisted laser desorption ionization time-of-flight mass spectrometry (MALDI-TOF), liquid chromatography-tandem mass spectrometry, isotope dilution mass spectrometry, size permeation (gel filtration), ion exchange, affinity, high performance liquid chromatography, ultra performance liquid chromatography, one-dimensional gel electrophoresis (1-DE), and/or two-dimensional gel electrophoresis (2-DE). In a certain embodiment, the kit comprises reagents for detecting the concentration of an amino acid adduct by liquid chromatography-tandem mass spectrometry. In a certain embodiment, the kit comprises reagents for detecting the concentration of an amino acid adduct by liquid chromatography-tandem mass spectrometry.

In some embodiments, the reagent comprises a chemical for detecting the concentration of an amino acid adduct in a sample by isotopic dilution analysis. In some embodiments, the reagent for quantifying NFK is [¹⁵N₂]NFK. In some embodiments, the reagent for quantifying DT is ring-[²H₆]DT. In some embodiments, the reagent for quantifying 3-NT is ring-[²H₃]3-NT. In some embodiments, the reagent for quantifying CEL is lysyl-[¹³C₆]CEL. In some embodiments, the reagent for quantifying CML is lysyl-[¹³C₆]CML. In some embodiments, the reagent for quantifying FL is lysyl-[¹³C₆]FL. In some embodiments, the reagent for quantifying CMA is carboxymethyl-[¹³C₂]CMA. In some embodiments, the reagent for quantifying G-H1 is guanidino [¹⁵N₂]G-H1. In some embodiments, the reagent for quantifying MG-H1 is guanidino-[¹⁵N₂]MG-H1. In some embodiments, the reagent for quantifying 3DG-H is guanidino-[¹⁵N₂]3DG-H. In some embodiments, the reagent for quantifying AASA is [²H₃]α-Aminoadipic acid. In some embodiments, the reagent for quantifying GSA is [2H₃]α-Aminoadipic acid. In some embodiments, the reagent for quantifying GSP is [¹³C₆]Glucosepane. In some embodiments, the reagent for quantifying pyrraline is [₁₃C₆, ¹⁵N₂] pyrraline. In some embodiments, the reagent for quantifying methylglyoxal-derived lysine dimer (MOLD) is [²H₈]MOLD, e.g. deuterium-MOLD. In any of the previous embodiments, alternative stable isotopic substitution is used in these compounds, as selected by a person of skill in the art and familiar with stable isotopic dilution analysis.

In some embodiments, the reagents for isotopic dilution analysis comprise at least one reagent selected from the group consisting of: [¹⁵N₂] NFK, ring-[²H₆]DT; ring-[²H₃]3-NT; lysyl-[¹³C₆]CEL, lysyl-[¹³C₆]CML, lysyl-[¹³C₆]FL, carboxymethyl-[¹³C₂]CMA, guanidino [¹⁵N₂]G-H1, guanidino-[¹⁵N₂]MG-H1, guanidino-[¹⁵N₂]3DG-H, [²H₃]α-Aminoadipic acid, [¹³C₆]Glucosepane, and [¹³C₆, ¹⁵N₂] pyrraline.

In some embodiments, the kits comprise a known quantity or concentration of the amino acid adducts described herein for use as a standard.

In some embodiments, the kit comprises instructions for carrying out the methods and uses of the invention as described herein. In certain embodiments, the kit comprises a software license or key to use software described herein, else said kit may comprise software described herein.

### EXAMPLES

### Materials & Methods

### Subject Recruitment

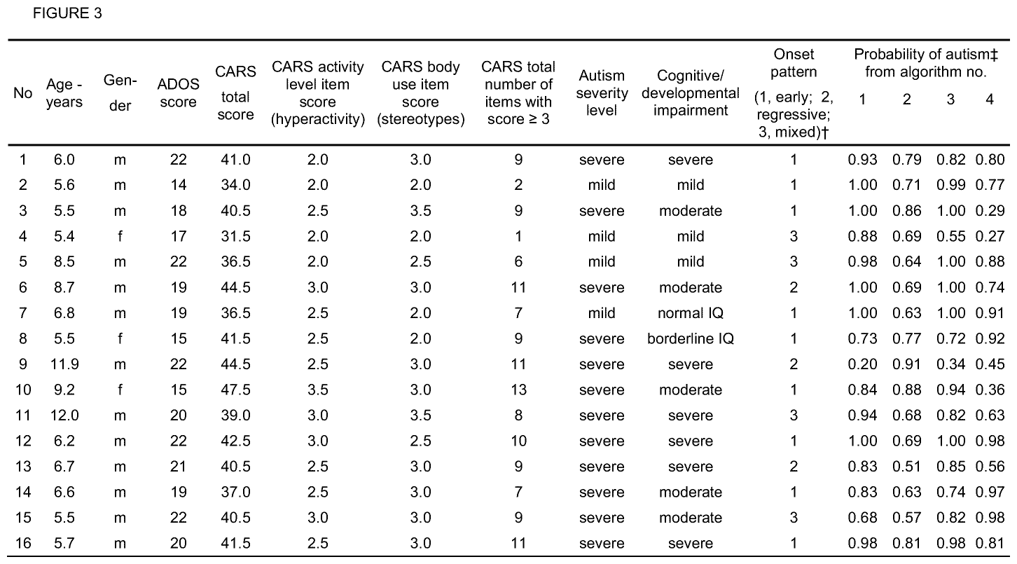

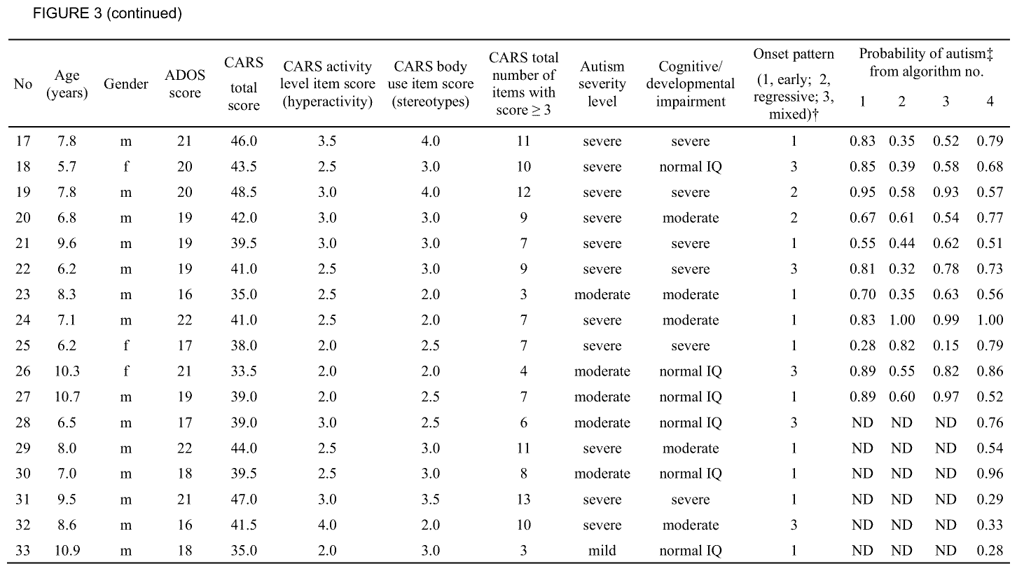

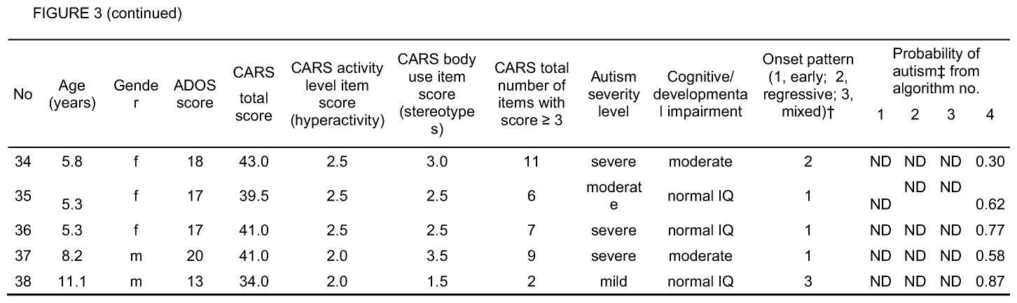

A total of 69 children were recruited. Of these, 38 had a diagnosis of ASD (29 males and 9 females) and 31 were classified as Typically Developing (TD) children (23 males and 8 females)—FIG. 2. The age of the two subject groups was not significantly different. Subject age was: ASD group, 7.6 years±2.0 years, range 5-12 years and TD group, 8.6±2.0 years, range 5-12 years. All ASD subjects received a diagnosis of ASD by two child development experts at the Child Neurology and Psychiatry Unit of the Bellaria Hospital of Bologna (IRCCS Institute of Neurological Sciences), according to the Diagnostic and Statistical Manual of Mental Disorders V (DSM 5 criteria, Autism Diagnostic Observation Schedule (ADOS), Childhood Autism Rating Scale (CARS) and characteristics of onset pattern of ASD previously defined. Developmental and cognitive levels were assessed by Psychoeducational Profile-3 (PEP-3) and Leiter International Performance Scale-Revised (Leiter-R). For both ASD and TD subjects, exclusion criteria were: presence of inflammatory or infective disease and taking antioxidant supplements at the time of study. No subject underwent any surgery intervention in the four months prior to blood and urine collection. None of the ASD subjects had active epilepsy at the time of blood and urine sampling. Subjects with ascertained medical and neurological comorbidity were excluded, through a medical work up including electroencephalography (recorded during awake and sleep), cerebral magnetic resonance imaging, standard clinical and neurological examination, neurometabolic and genetic investigations (including comparative genomic hybridization array, molecular assay for Fragile X and MECP2). Subjects recruited for this study were not taking any medication. TD children were recruited in the local community, with no sign of cognitive, learning and psychiatric involvement. They were attending mainstream school and had not been subjected to stressful events. Dietary habits were assessed by a Food Questionnaire, built according to the guidelines issued by the Emilia-Romagna Health Authority. No ASD child was on a diet free of gluten or casein. Both patients and controls were on a typical Mediterranean diet, as defined by the prevalence of both simple and complex carbohydrates, use of olive oil, and plenty of fruit. The consumption of vegetables was less than desirable in both patients and controls, although vegetable intake was more limited in ASD patients. Demographic and clinical features of ASD are summarized in FIG. 3. All subjects were recruited at the Child Neurology and Psychiatry Unit of the Bellaria Hospital of Bologna, Bologna, Italy.

Thirty-eight children with ASD were recruited for this study. The distribution of severity of ASD in this subject group recruited was (number of cases): mild (6), moderate (6) and severe (26). The distribution of cognitive/developmental impairment was (number of cases): normal/borderline IQ (11), mild (3), moderate (12) and severe (12). The distribution of onset pattern of ASD was (number of cases): early (22), regressive (6) and mixed (10). The ADOS score ranged from 13 to 22 and the CARS total score from 31.5 to 48.5.

### Blood and Urine Sampling

Blood was withdrawn in the morning from fasting children. Spot urine samples were the first ones in the morning. Blood samples were collected using ethylenediaminetetra-acetic acid (EDTA) as anticoagulant. Plasma and blood cells were separated immediately by centrifugation (2000 g, 10 min) and plasma samples stored at −80° C. until analysis and transferred between collaborating laboratories on dry ice.

### Assay of Markers of Protein Glycation, Oxidation and Nitration

The content of glycated, oxidized and nitrated adduct residues in plasma protein was quantified in exhaustive enzymatic digests by stable isotopic dilution analysis liquid chromatography-tandem mass spectrometry (LC-MS/MS), with correction for autohydrolysis of hydrolytic enzymes. The concentrations of related glycated, oxidized and nitrated amino acid free adducts (glycated, oxidized and nitrated amino acids) in plasma and urine were determined similarly in plasma and urine ultrafiltrate, respectively. Ultrafiltrate of plasma (50 μl) was collected by microspin ultrafiltration (10 kDa cut-off) at 4° C. Retained protein was diluted with water to 500 μl and washed by 4 cycles of concentration to 50 μl and dilution to 500 μl with water over the microspin ultrafilter at 4° C. The final washed protein (100 μl) was delipidated and hydrolyzed enzymatically as described (Rabbani et. al. Biochem Soc Trans. 2014; 42 (2): 511-7; Ahmed et. al. Sci Rep. 2015; 5:9259). Ultrafiltrate of urine (50 μl) was collected by microspin ultrafiltration (3 kDa cut-off) at 4° C.

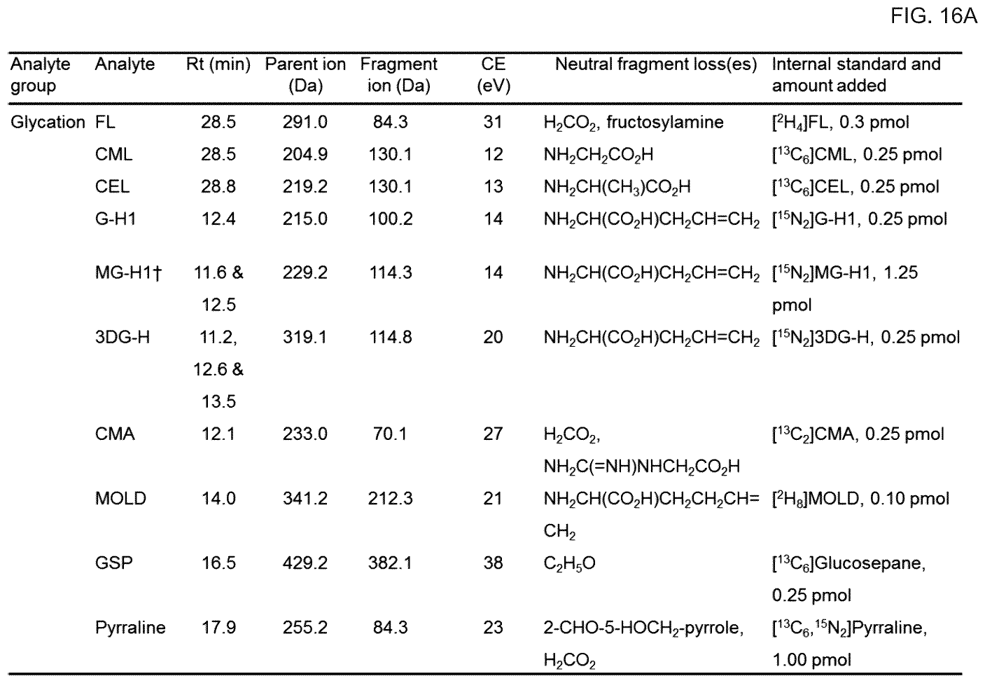

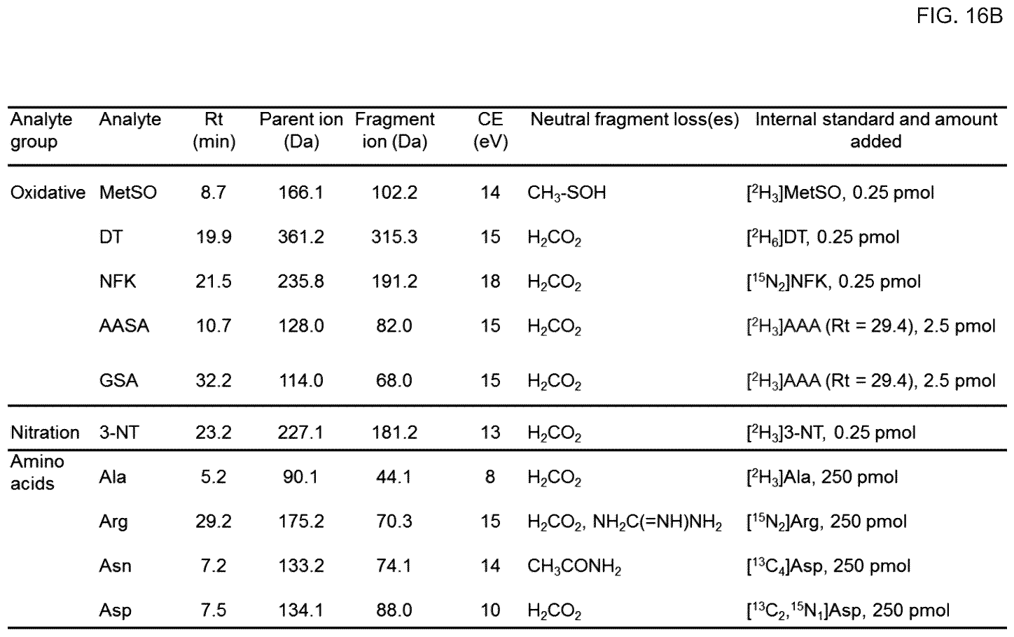

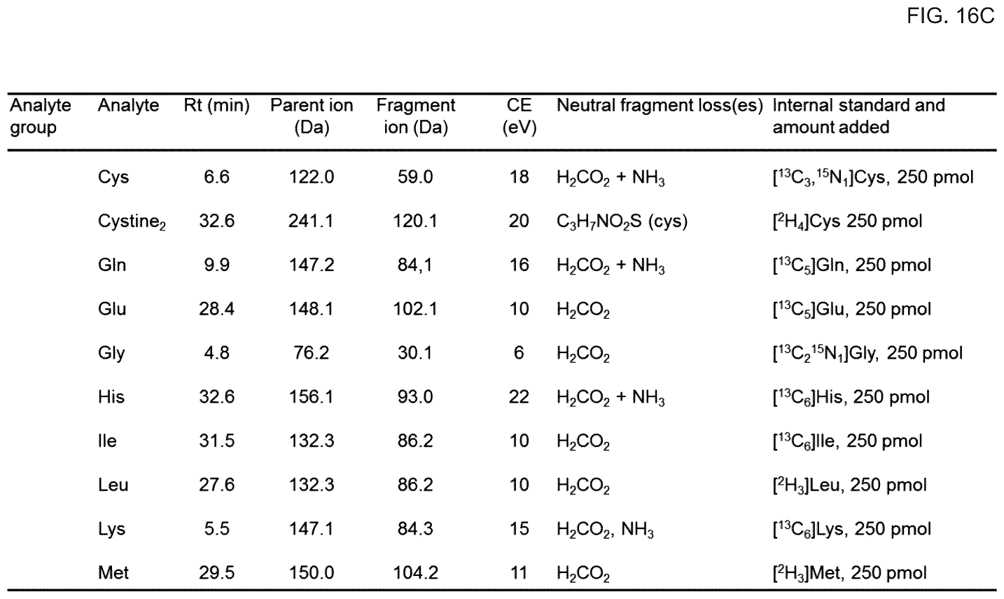

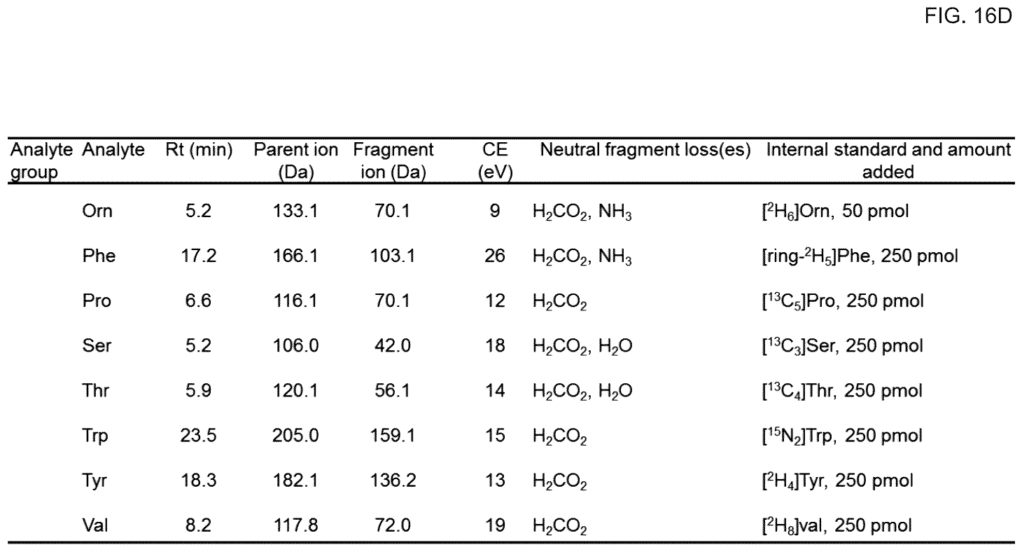

Protein hydrolysate (25 μl, 32 μg equivalent) or ultrafiltrate (5 μl) was mixed with stable isotopic standard analytes and analyzed by LC-MS/MS using an Acquity™ UPLC system with a Xevo-TQS tandem mass spectrometer (Waters, Manchester, U.K.). Samples are maintained at 4° C. in the autosampler during batch analysis. The columns were: 2.1×50 mm and 2.1 mm×250 mm, 5 μm particle size Hypercarb™ (Thermo Scientific), in series with programmed switching, at 30° C. Chromatographic retention was used to resolve oxidized analytes from their amino acid precursors to avoid interference from partial oxidation of the latter in the electrospray ionization source of the mass spectrometric detector. Analytes were detected by electrospray positive ionization and mass spectrometry multiple reaction monitoring (MRM) mode where analyte detection response was specific for mass/charge ratio of the analyte molecular ion and major fragment ion generated by collision-induced dissociation in the mass spectrometer collision cell. The ionization source and desolvation gas temperatures were 120° C. and 350° C., respectively, cone gas and desolvation gas flow rates were 99 and 900 l/h and the capillary voltage was 0.60 kV. Argon gas (5.0×10-3 mbar) was in the collision cell. For MRM detection, molecular ion and fragment ion masses and collision energies optimized to ±0.1 Da and ±1 eV, respectively, were programmed-FIG. 16.

Analytes determined were: glycation adducts-FL, and AGEs, CML, CEL, pyrraline, CMA, G-H1, MG-H1, 3DG-H, MOLD and GSP; oxidation adducts-DT, NFK, AASA, GSA; nitration adduct, 3-NT; and all major amino acids.

Oxidation, nitration and glycation adduct residues are normalized to their amino acid residue precursors and given as mmol/mol amino acid modified; and related free adducts are given in nM. Chemical structures and biochemical and clinical significance of these analytes have been described elsewhere (Thornalley & Rabbani, Biochim Biophys Acta. 2014; 1840 (2): 818-29; and Ahmed et. al, Sci Rep. 2015; 5 (9259): 9251-7). Renal clearance (CL) of glycation, oxidation and nitration free adducts and unmodified amino acids was deduced from plasma and spot urine collections: CL (μl/mg creatinine or ml/mg creatinine)=[Analyte]Urine (nmol/mg creatinine)/[Analyte]Plasma (pmol/ml or nmol/ml).

### Machine Learning Analysis

The objective was to distinguish between children with ASD and healthy controls. In all cases, the diagnostic algorithms were trained on 50% of the cases and controls (training subset) before being used to predict the disease class for each sample in the remaining subjects (test set); 2-fold cross-validation. The outcome was to assign, for each test set sample, a set of probabilities corresponding to each of the ASD/control groups—the group assignment being that for which the probability is highest. Test data were held separate from algorithm training; algorithm settings were not adjusted once analysis of the test set data began-thereby guarding against overfitting and hence providing a rigorous estimate of predictive performance.

Four algorithm types were tested for performance: Random Forests, logistic regression, ensemble classifier, and Support Vector Machines (SVMs).

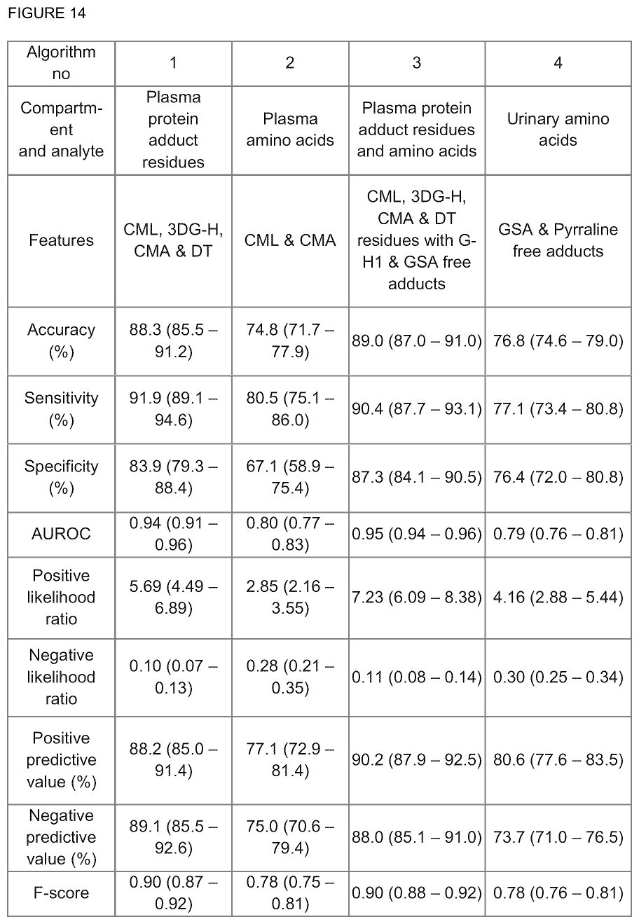

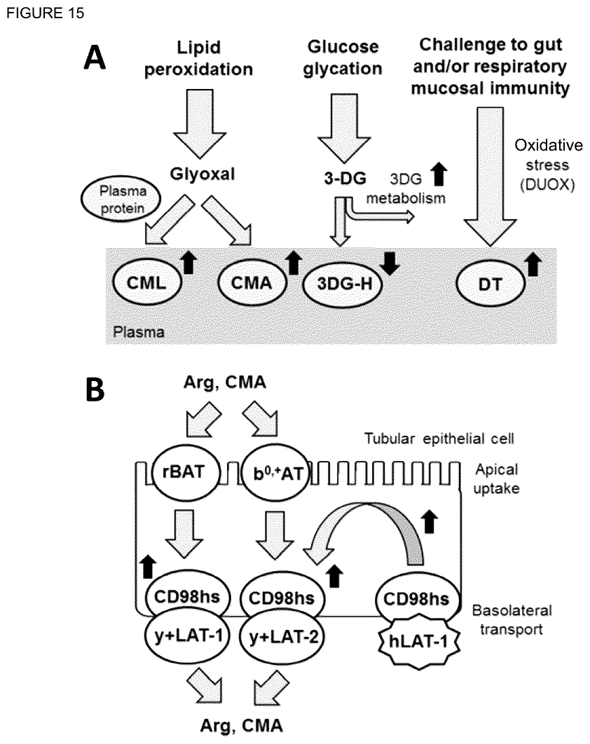

During the algorithm training, the complete panel of protein glycation, oxidation and nitration adducts were used as features and developed algorithms for each analyte type: plasma protein adduct residues, plasma free adducts and urinary free adducts. For the latter two, unmodified amino acids were also included as features. The aim during the training was to select the set of features that accomplishes the highest performance. The machine learning experiments were initially explored using all metabolite features. Advantageously, subsequent selection of a subset of discriminant biomarker features improved the algorithm performance (see FIG. 14). For the biomarker (e.g. amino acid adduct) selection, a sequential feature selection approach was used. The biomarker feature selection and classifier selection were made on the basis of algorithm performance defined by classification accuracy, sensitivity, specificity, area under-the-curve of the receiver operating characteristic curve (AUROC), positive likelihood ratio, negative likelihood ratio, positive predictive value, negative predictive value, and F-measure. For each performance metric, the mean and 95% CI was determined and reported. The algorithm training and testing was repeated 10 times, without altering the algorithm parameters, with 50% data split, to test for algorithm's robustness against any bias towards data split. Computer programs were developed using Statistics and Machine Learning Toolbox of MATLAB® (MathWorks, Inc., Natick, USA), with a linear kernel SVM and sequential minimal optimization (SMO).

### Statistical Analysis

Data are presented as mean±SD for parametric distributions and median (lower-upper quartile) for non-parametric distributions. The test for normality of data distribution applied was the Kolmogorov-Smirnov test. Significance was evaluated by Student's t-test or by Mann-Whitney U-test for parametrically or non-parametrically distributed data, respectively. Bonferroni correction was made for analysis of multiple analytes without preconceived hypothesis. Correlation analysis was performed by the Spearman's rho method with continuous variables. For clinical categorical variables with ≥6 categories, Spearman correlation was performed-assuming approximation to a continuous variable; for other categorical variables, significance of difference of biomarker data distributions between categories was assessed by one-way ANOVA for parametric data and Kruskal-Wallis H test. Data were analyzed using SPSS, version 24.0.

For power analysis in the study design, the level of the irreversible oxidative damage marker DT in plasma protein was chosen. In healthy human subjects, plasma protein DT was 0.0287±0.0027 mmol/mol tyr (n=29) in previous studies. This study was designed to detect a 50% increase in plasma protein DT at the 0.01% significance level, for which ≥18 case and control samples were required. Post-hoc analysis revealed an 88% increase with P=0.00017, after Bonferroni correction of 14 with 27 cases and 21 controls, suggesting the study was adequately powered for this key target analyte.

### Example 1

Children with Autistic Spectrum Disorder Recruited for this Study

Thirty-eight children with ASD were recruited for this study. The distribution of severity of ASD in this subject group recruited was (number of cases): mild (6), moderate (6) and severe (26). The distribution of cognitive/developmental impairment was (number of cases): normal/borderline IQ (11), mild (3), moderate (12) and severe (12). The distribution of onset pattern of ASD was (number of cases): early (22), regressive (6) and mixed (10). The ADOS score ranged from 13 to 22 and the CARS total score from 31.5 to 48.5.

### Example 2

### Plasma Protein Glycation, Oxidation and Nitration

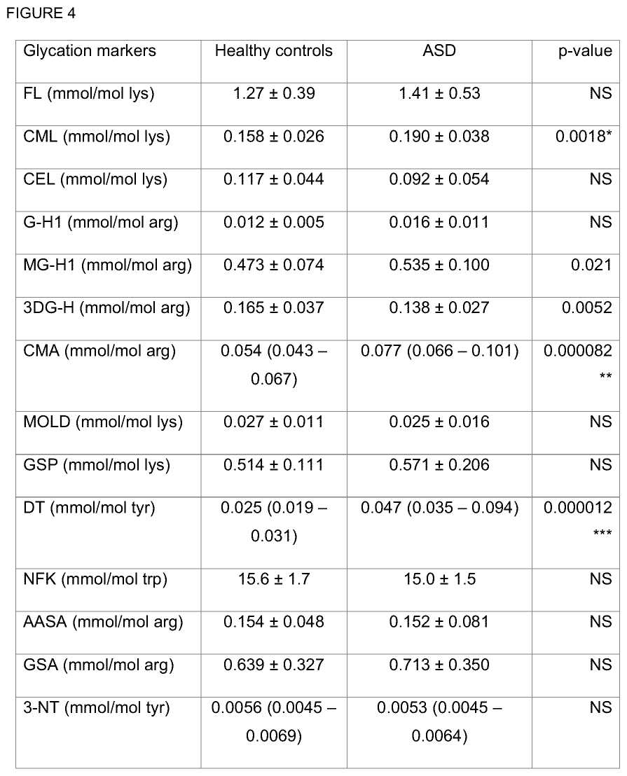

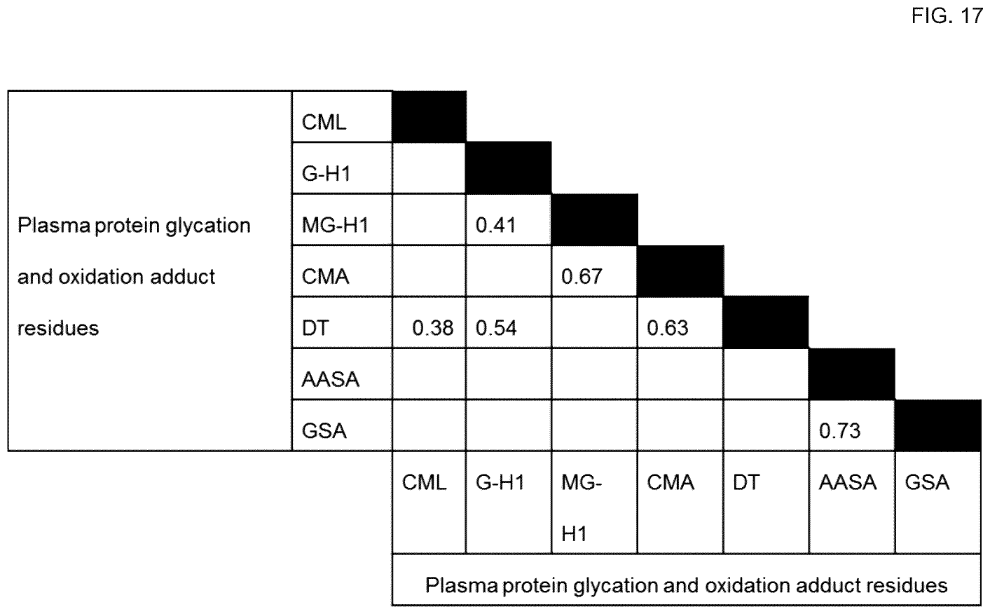

In plasma protein, protein content of AGEs-CML, MG-H1 and CMA-were increased in children with ASD, with respect to healthy controls; whereas plasma protein content of AGE, 3DG-H, was decreased in children with ASD, with respect to healthy controls. Plasma protein content of the oxidative damage adduct, DT, was increased in children with ASD, with respect to healthy controls. Advantageously, changes in CML, CMA and DT remained significant after Bonferroni correction for measurement of multiple analytes (FIG. 4). In correlation analysis, highly significant positive correlations (P<0.01, Spearman) were of CML with DT, G-H1 with MG-H1 and DT, MG-H1 with CMA, CMA with DT, and AASA with GSA-FIG. 17. No correlation or association of glycation, oxidation and nitration adduct residues was found with demographic and clinical features. There was no significant difference of these variables between subject groups of different genders with and without ASD.

### Example 3

### Plasma Glycated, Oxidized and Nitrated Amino Acids and Amino Acid Metabolome

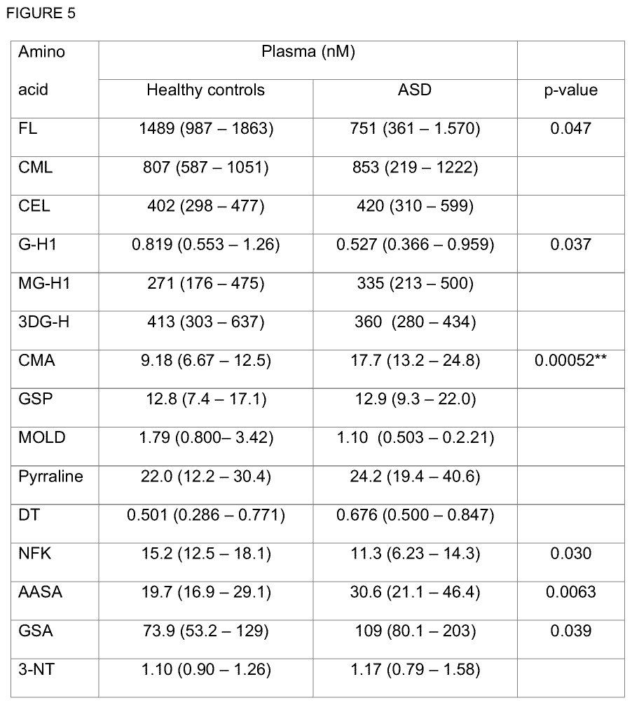

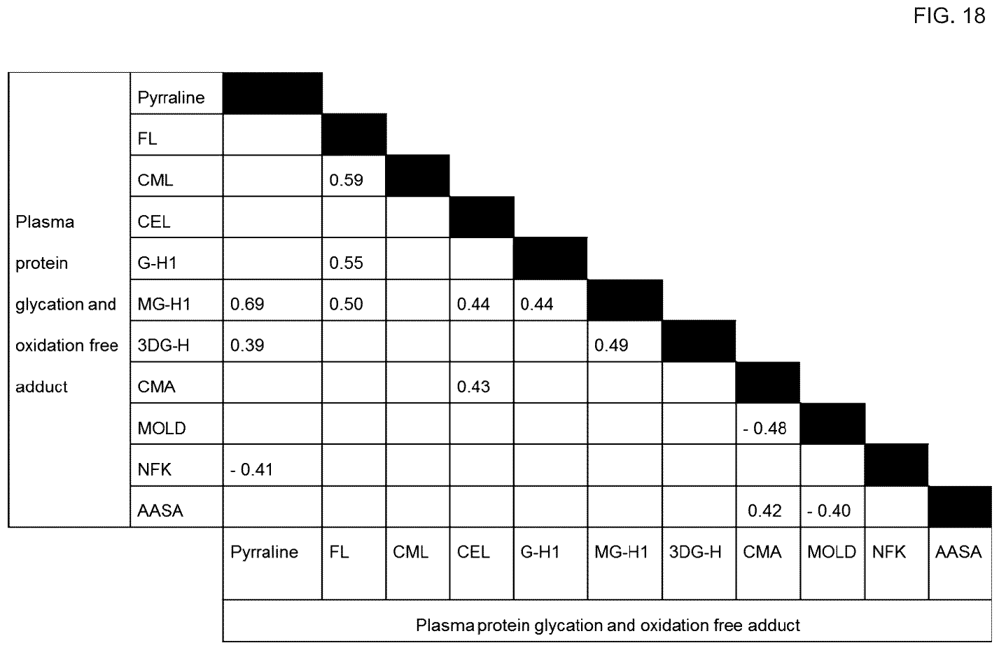

For glycated, oxidized and nitrated amino acid concentration in plasma, FL, G-H1 and NFK were decreased whereas CMA, AASA and GSA were increased in children with ASD, with respect to healthy controls. Advantageously, increase in CMA remained significant after Bonferroni correction (FIG. 5). In correlation analysis, highly significant positive correlations were of pyrraline with MG-H1 and 3DG-H, FL with CML, G-H1 and MG-H1, CEL with MG-H1 and CMA, MG-H1 with 3DG-H, and CMA with AASA. There were highly significant negative correlations of pyrraline with NFK, CMA with MOLD, and MOLD with AASA-FIG. 18.

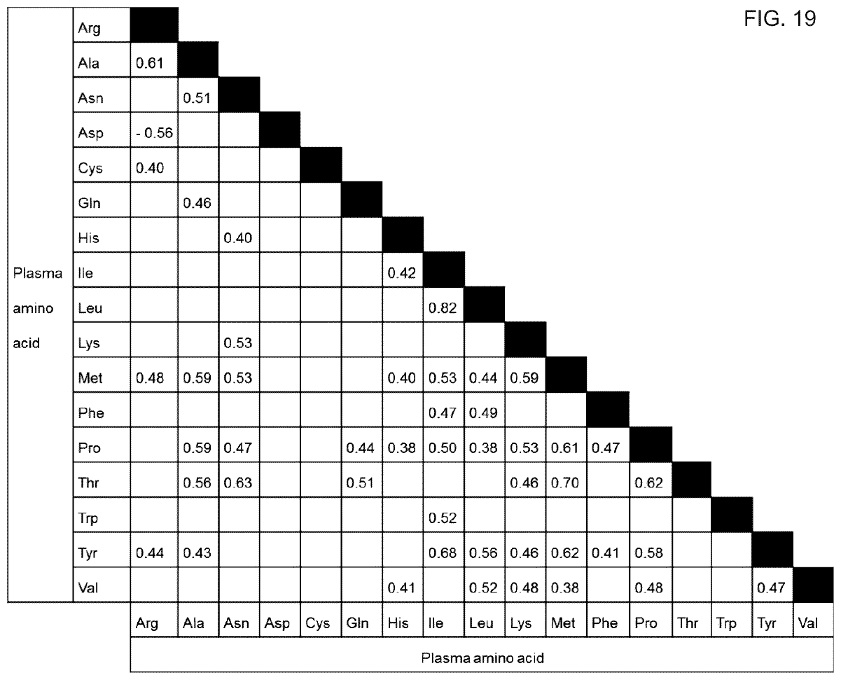

For the conventional amino acid metabolome, there were increases in arg, gln, glu and thr and decrease in trp in children with ASD, with respect to healthy controls. There were many highly significant positive correlations between plasma amino acid concentrations-FIG. 19. No correlation or association of glycation, oxidation and nitration free adducts and amino acids was found with demographic and clinical features. There was no significant difference of these variables between genders

### Example 4

### Urinary Glycated, Oxidized and Nitrated Amino Acids and Amino Acid Metabolome and Renal Clearance

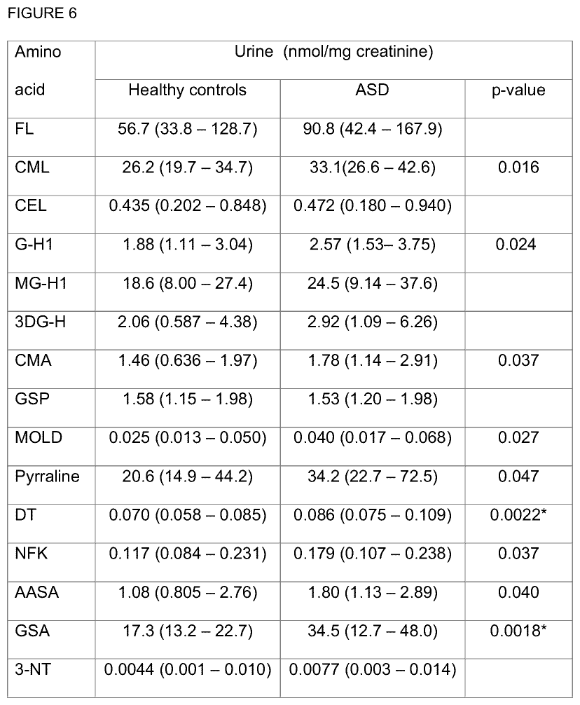

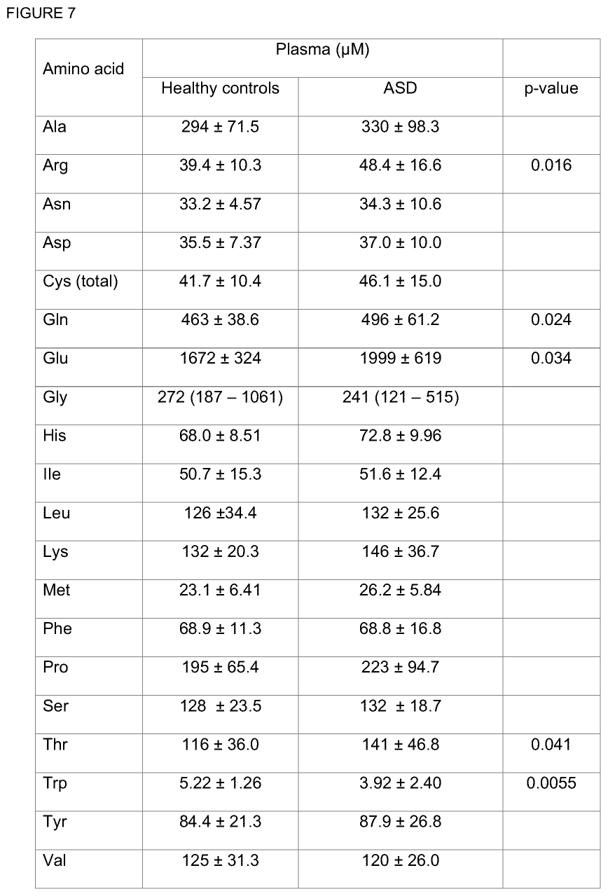

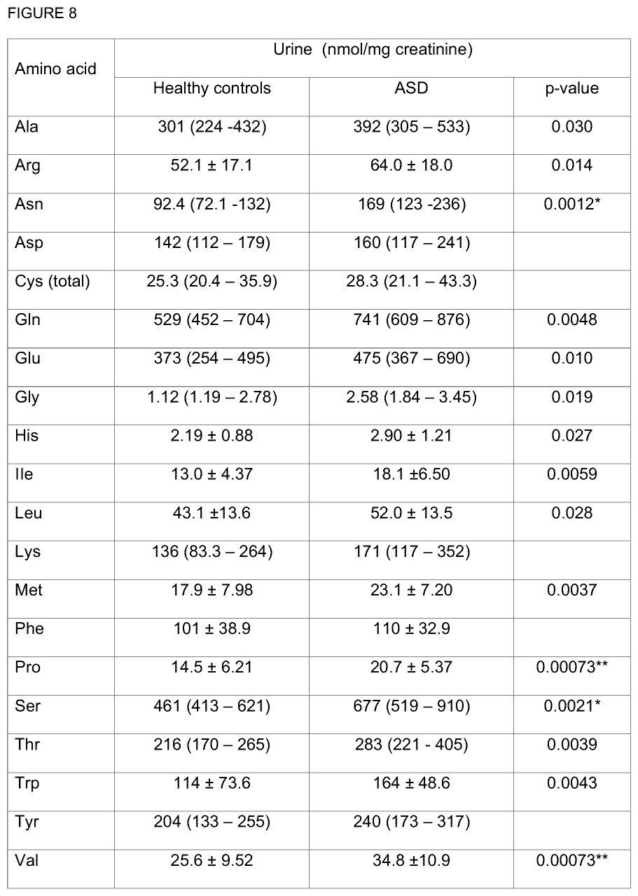

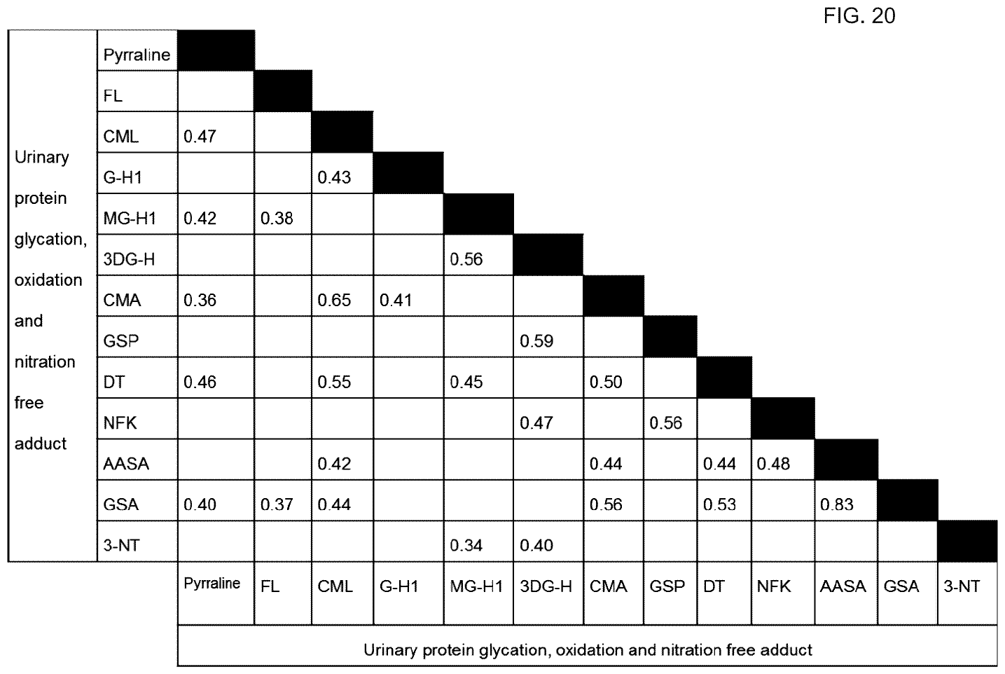

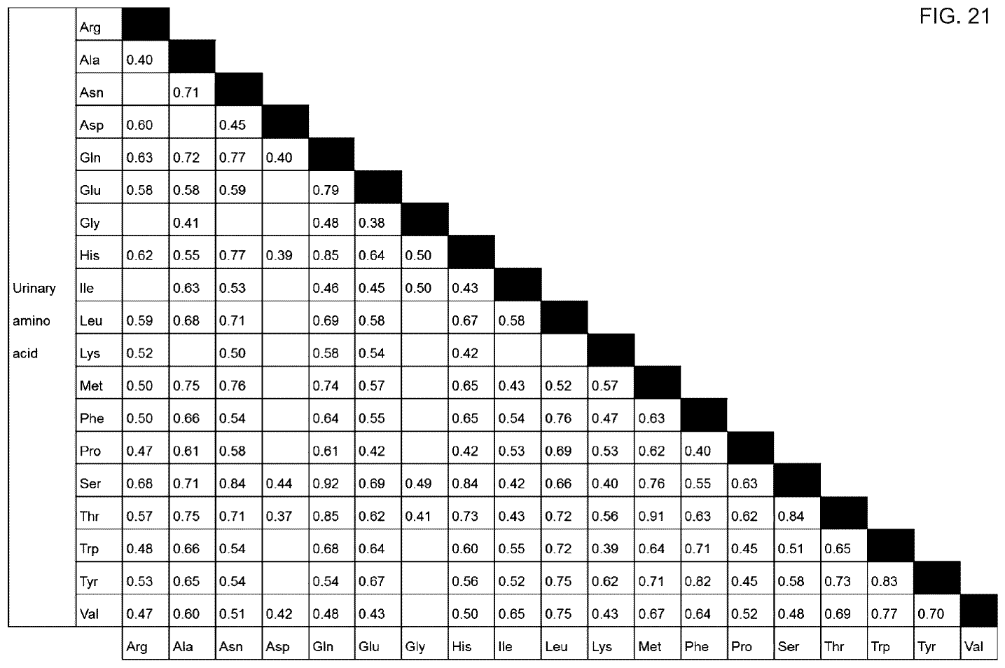

For the urinary flux of glycated, oxidized and nitrated amino acids, children with ASD showed increased urinary excretion of CML, G-H1, CMA, MOLD, pyrraline, DT, NFK, AASA and GSA. Advantageously, urinary excretions of DT and GSA remained significant after Bonferroni correction (FIG. 6). For the urinary flux of unmodified amino acids, children with ASD showed increased urinary excretion of all amino acids except asp, cys, lys, phe and tyr. Advantageously, increases in urinary excretion of asn, pro, ser and val remained significant after Bonferroni correction (FIG. 8). There were several highly significant positive correlations between urinary excretions of glycation, oxidation and nitration adducts and amino acids-see FIGS. 20 and 21.

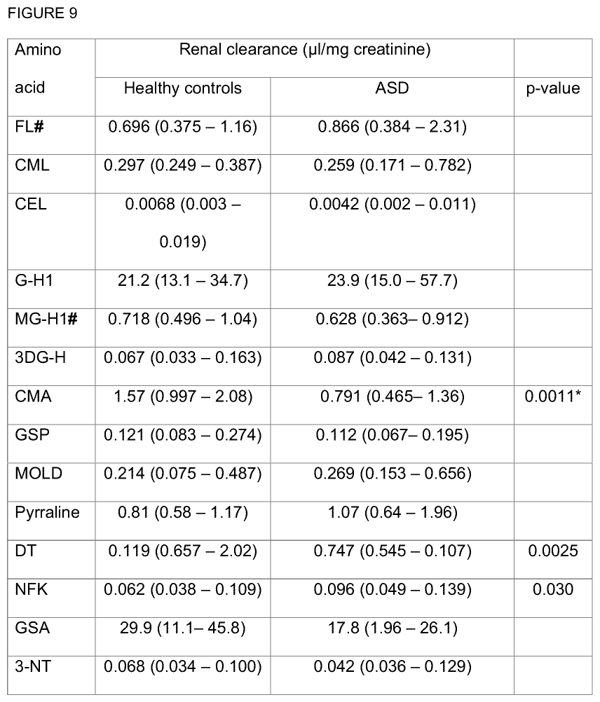

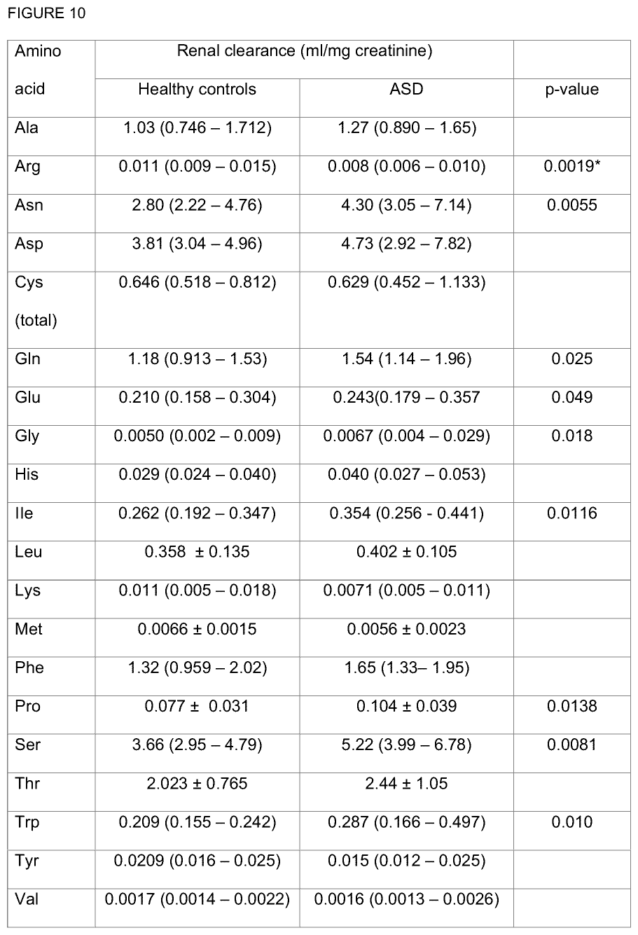

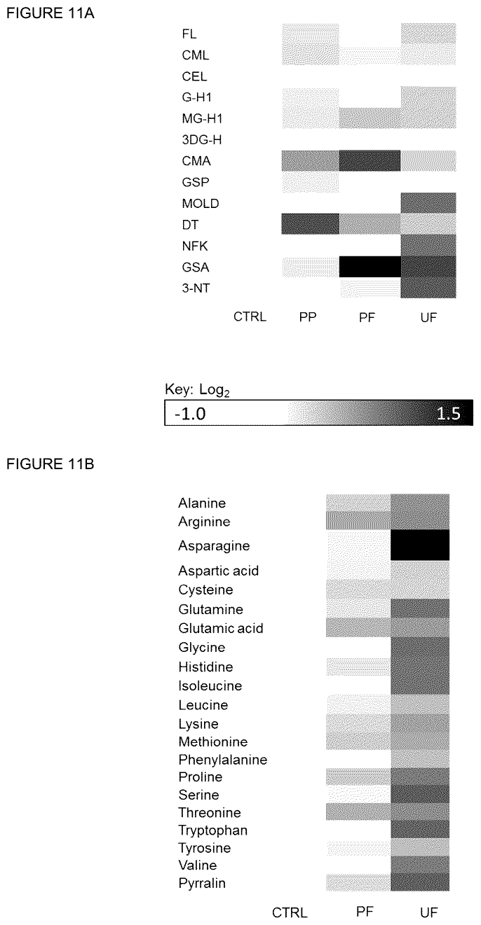

Renal clearance of CMA, GSP, DT, arg, glu, leu, phe and thr were decreased and renal clearance of NFK and trp were increased in children with ASD, with respect to healthy controls. Advantageously, decreases in renal clearance of arg and CMA remained significant after Bonferroni correction: CLarg decreased 32% and CLCMA decreased 50% in children with ASD, compared to healthy control; P<0.001 (FIGS. 9 and 10). No correlation or association of these glycation, oxidation and nitration free adduct and amino acid variables was found with demographic and clinical features. There was no significant difference of these variables between genders.

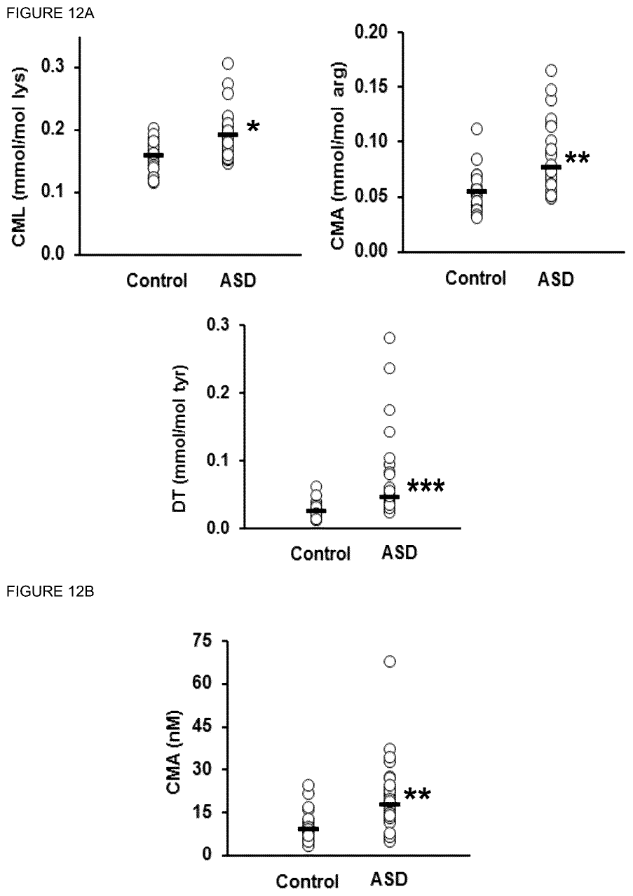

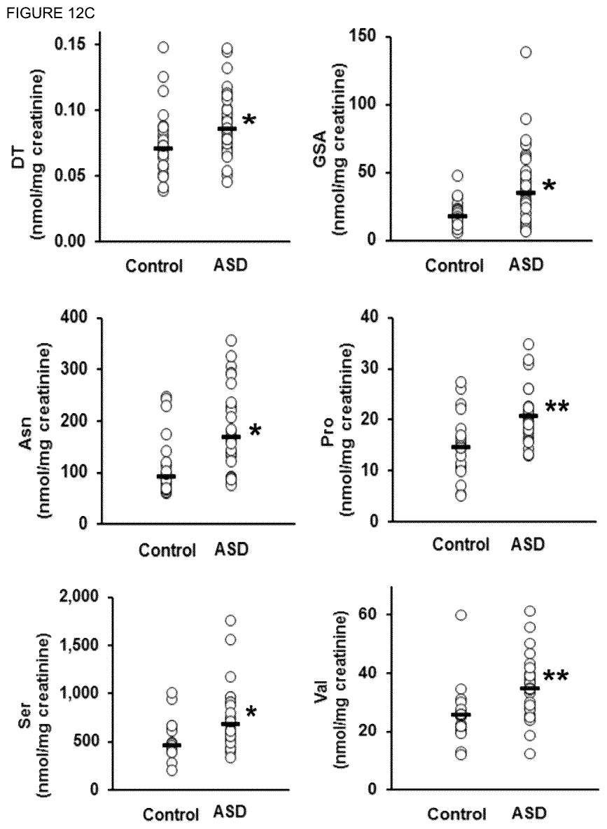

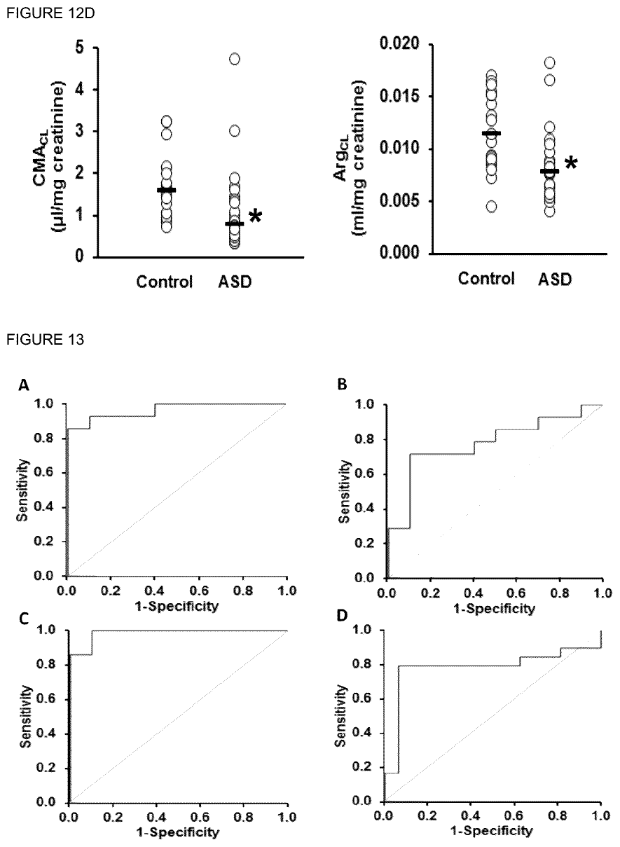

Changes of glycation, oxidation and nitration adducts and amino acid metabolome in plasma and urine are summarized in heat maps (FIGS. 11A and 11B). Data distributions of biomarker (amino acid adduct and amino acid) with significantly different change in the ASD study group after Bonferroni correction are given in FIG. 12.

### Example 5

### Development of Diagnostic Algorithms for Autistic Spectrum Disorder

To explore diagnostic utility of protein glycation, oxidation and nitration measurements for ASD, we analyzed plasma and urinary amino acid analyte data by a machine learning approach. SVMs was the best-performing method out of the four algorithms that were investigated. Algorithm optimized from 2-fold cross-validation were as below.

Algorithm-1, developed from plasma protein glycation, oxidation and nitration adduct residue analytes.

It has the following features: CML, 3DG-H, CMA and DT. Classification accuracy was 88%, sensitivity 92%, specificity 84% and AUROC 0.94. A random outcome is 0.50.

Algorithm-2, developed from plasma glycated, oxidized and nitrated amino acids and conventional amino acid metabolome.

It has the following features: CML and CMA. Classification accuracy was 75%, sensitivity 81% and specificity 67% and AUROC 0.80.

Algorithm-3, developed from plasma protein glycation, oxidation and nitration adduct residues and plasma glycated, oxidized and nitrated amino acids and conventional amino acid metabolome combined.

It has the following features: plasma protein CML, 3DG-H, CMA and DT residues and plasma G-H1 and GSA free adducts. Classification accuracy was 89%, sensitivity 90%, specificity 87% and AUROC 0.95.

Algorithm-4, developed from urinary glycated, oxidized and nitrated amino acids.

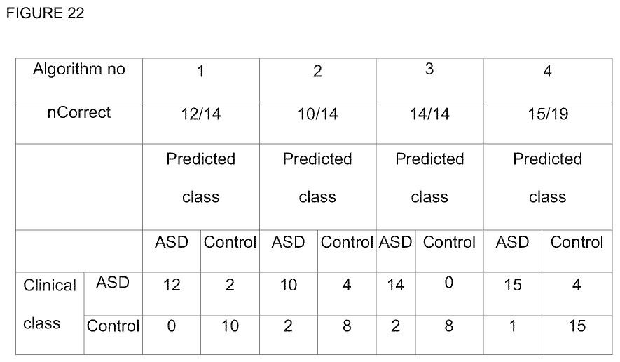

It has the following features: GSA and pyrraline free adducts. Classification accuracy was 77%, sensitivity 77%, specificity 76% and AUROC 0.79 (FIGS. 13, 14 and 22).

The diagnostic algorithms were used to deduce the probability of having ASD for each patient diagnosed with ASD by clinical symptoms (FIG. 3). The association and correlation of these probabilities with clinical features was explored. No significant association or correlation of these probabilities with clinical features (age, ADOS, total CARS, CARS hyperactivity and CARS body use scores, autism severity, cognitive/developmental impairment and ASD onset pattern) was found.

Without wishing to be bound by theory, the findings of the present inventors implicate a disturbance of metabolism of dicarbonyl precursors of advanced glycation end products (AGEs) and activation of dual oxidase (DUOX) in ASD. The initial evidence given herein suggests detection of the combination of plasma protein AGE and dityrosine (DT) concentrations may provide an optimal blood-based test for diagnosis of ASD. Decreased renal clearance of arginine and CMA is proposed to be linked to amino acid transporter dysfunction in ASD, building on increasing evidence of neuronal amino acid availability as a driver in ASD development.

### Example 6

In the recent validation study, we found a positive correlation of the contents of two modified amino acid residues of plasma protein with severity of ASD as assessed by Autism Diagnostic Observation Schedule-2 (ADOS-2) score. The modified amino acids were: methylglyoxal-derived hydroimidazolone MG-H1 and methylglyoxal-derived Ne (1-carboxyethyl) lysine (CEL). As both adducts are derived from methylglyoxal, it may be suggested that severe ASD is linked to exposure to increased levels of the reactive metabolite methylglyoxal and speculated that pharmacological agents that decrease methylglyoxal may alleviate severe symptoms of ASD.

All publications mentioned in the above specification are herein incorporated by reference. Various modifications and variations of the described methods and system of the present invention will be apparent to those skilled in the art without departing from the scope and spirit of the present invention. Although the present methods have been described in connection with specific preferred embodiments, it should be understood that the methods as claimed should not be unduly limited to such specific embodiments. Indeed, various modifications of the described modes for carrying out the methods which are obvious to those skilled in biochemistry and biotechnology or related fields are intended to be within the scope of the following claims.

1. A method for diagnosing an autistic spectrum disorder (ASD), said method comprising:
a. obtaining a sample from a subject;
b. detecting a concentration of one or more amino acid adducts in the sample obtained from a subject;
c. calculating a probability of the subject having ADS; and
d. diagnosing the patient.

2. The method of claim 1, wherein calculating the probability of the subject having ADS utilizes a diagnostic algorithm.

3. The method of claim 2, wherein the diagnostic algorithm is selected from a classification, a support vector machine, a decision tree, a regression model, a neural network, a random forest algorithm, or any combination thereof.

4. The method of claim 2, wherein the diagnostic algorithm uses a machine vector approach.

5. The method of claim 4, wherein the machine vector approach further comprises a two fold cross-validation approach.

6. The method of claim 5, wherein the diagnostic algorithm is trained on a population of subjects known to have ASD.

7. The method of claim 5, wherein the diagnostic algorithm is trained on a population of subjects known to not have ASD.

8. The method of claim 2, wherein the amino acid adducts are detected in plasma, urine, or any combination thereof.

9. The method of claim 1, further comprising determining the severity of ASD in a subject.

10. A method for determining prognosis of an autistic spectrum disorder (ASD), said method comprising:
a. detecting the concentration of an amino acid adduct in a sample obtained from a subject, wherein said amino acid adduct is a glycated amino acid adduct, an oxidised amino acid adduct, or a nitrated amino acid adduct present in the sample as a free amino acid and/or amino acid residue of sample protein;
b. comparing the concentration of the amino acid adduct in the sample with the concentration of the same amino acid adduct in a reference standard; and
c. identifying the presence or absence of a concentration difference of said amino acid adduct in the sample relative to the reference standard;
wherein the presence or absence of a concentration difference correlates with a good prognosis or a poor prognosis.

11.-49. (canceled)
